(19) United States
(12) Patent Application Publication
(10) Pub. No.: US 2022/0287957 A1
(43) Pub. Date: Sep. 15, 2022

(54) COMPOSITIONS AND METHODS FOR USING ALTERNATING ELECTRIC FIELDS TO DISRUPT NANOPARTICLES

(71) Applicant: NOVOCURE GMBH, Root (CH)

(72) Inventor: Preshita Prafulla Desai, Pomona, CA (US)

(21) Appl. No.: 17/692,997

(22) Filed: Mar. 11, 2022

Related U.S. Application Data
(60) Provisional application No. 63/160,476, filed on Mar. 12, 2021.
(60) Provisional application No. 63/216,940, filed on Jun. 30, 2021.

(51) Int. Cl. A61K 9/00; A61N 1/40; A61K 31/7068; A61P 35/00; A61K 9/51

(52) U.S. Cl. CPC A61K 9/0009; A61N 1/40; A61K 31/7068; A61P 35/00; A61K 9/5146; A61K 9/5161; A61K 9/5169

(57) ABSTRACT

Disclosed are methods comprising: administering a drug-loaded nanoparticle to a subject; and exposing a target site of the subject to an alternating electric field for a period of time, the alternating electric field having a frequency and field strength, and wherein the alternating electric field triggers the release of the drug from the drug-loaded nanoparticle at the target site of the subject. Disclosed are methods comprising: administering a drug-loaded nanoparticle to a subject; and exposing a target site of the subject to an alternating electric field for a period of time, the alternating electric field having a frequency and field strength, wherein the alternating electric field triggers the release of the drug from the drug-loaded nanoparticle at the target site of the subject, thereby decreasing non-target site specific release of the drug. Disclosed are methods of treating cancer in a subject comprising administering a drug-loaded nanoparticle to a subject having cancer; and exposing a target site of the subject to an alternating electric field for a period of time, the alternating electric field having a frequency and field strength, wherein the target site comprises a cancer cell; wherein the alternating electric field triggers the release of the drug from the drug-loaded nanoparticle at the target site of the subject; wherein the drug kills the cancer cell. Disclosed are methods of killing cancer cells comprising administering a drug-loaded nanoparticle to a subject; and exposing a target site of the subject to an alternating electric field for a period of time, the alternating electric field having a frequency and field strength, wherein the alternating electric field triggers the release of the drug from the drug-loaded nanoparticle at the target site of the subject, wherein the target site comprises cancer cells, wherein the drug kills the cancer cells.

### CROSS-REFERENCE TO RELATED APPLICATIONS

This application claims the benefit of U.S. Provisional Patent Application No. 63/160,476, filed on Mar. 12, 2021, and U.S. Provisional Patent Application No. 63/216,940, filed on Jun. 30, 2021, each of which is incorporated by reference herein in its entirety.

### BACKGROUND

Tumor Treating Fields (TTFields), or alternating electric fields, have emerged as a promising, safe, non-invasive modality with minimal to no side effects (limited to skin) to treat cancer. After the FDA approval and proven success in treatment glioblastoma multiforme, TTFields have been extensively investigated for their efficacy in other cancers like pancreatic cancer (PC), ovarian cancer, non-small cell lung cancer etc. and is currently under progressive clinical trials (1-7).

PC is one amongst the most challenging cancers to treat. Currently, PC afflicts over 55,000 Americans and claims over 45,000 lives every year (˜7% of cancer related deaths). The PC incidence is expected to rise by ˜55% in coming years making it the second leading cause of cancer related deaths (8, 9). In spite of the extensive research leading to the newer promising approaches like multidrug treatment, surgical neoplasm resection, advanced radiation etc., the 5 year survival rate of PC patients is still retained at less than 5-7% (8, 9). Further, PC recurrence rate is very high after partial/complete remission and is extremely unlikely to be cured (10, 11). Furthermore, the existing treatment modalities offer severe adverse effects that gravely compromise the patient's quality of life. This clearly depicts the pressing need to find alternative approaches to treat and prevent PC which are more effective as well as more patient tolerable.

Specifically, in the area of PC, TTFields (150 kHz) alone and in combination with anticancer drugs have demonstrated promising in vitro antiproliferative effect in multiple PC cell lines and in vivo studies have confirmed decrease in tumor volume with delay in tumor growth. The clinical studies in this direction have established acceptable safety profile and the Phase II study PANOVA (NCT01971281) in combination with existing PC drug treatment Gemcitabine and nab-Paclitaxel have demonstrated clinical promise. The studies showed significant increase in ‘progression free survival’ and ‘overall survival’ of PC patients with 2-fold increase in 1 year survival rate. Based on this, Phase III PANOVA-3 trial is in progress (2, 5, 12). This clearly confirms efficacy of TTFields in PC treatment alone and in combination with anticancer drugs.

Importantly, increased efficacy, high tolerability and low adverse effects are the critical pillars in success of TTFields as a cancer treatment modality (5). However, it is important to note that use of TTFields with anticancer drugs will continue to offer non-site specific severe adverse effects associated with anticancer drugs which in turn will continue to compromise the patient's life. Hence, there is an unmet need in researching the ways to reduce the adverse effects elicited by the concurrent use of anticancer drugs with TTFields.

### BRIEF SUMMARY

Disclosed are methods and compositions for using alternating electric fields to burst nanoparticles allowing for the release of a therapeutic from inside the nanoparticle.

Disclosed are methods of triggering or initiating drug release at a target site of a subject comprising: administering a drug-loaded nanoparticle to a subject; and exposing a target site of the subject to an alternating electric field for a period of time, the alternating electric field having a frequency and field strength, and wherein the alternating electric field triggers the release of the drug from the drug-loaded nanoparticle at the target site of the subject.

Disclosed are methods of decreasing non-target site specific release of a drug in a subject comprising: administering a drug-loaded nanoparticle to a subject; and exposing a target site of the subject to an alternating electric field for a period of time, the alternating electric field having a frequency and field strength, wherein the alternating electric field triggers the release of the drug from the drug-loaded nanoparticle at the target site of the subject, thereby decreasing non-target site specific release of the drug.

Disclosed are methods of treating cancer in a subject comprising administering a drug-loaded nanoparticle to a subject having cancer; and exposing a target site of the subject to an alternating electric field for a period of time, the alternating electric field having a frequency and field strength, wherein the target site comprises a cancer cell; wherein the alternating electric field triggers the release of the drug from the drug-loaded nanoparticle at the target site of the subject; wherein the drug kills the cancer cell.

Disclosed are methods of killing cancer cells comprising administering a drug-loaded nanoparticle to a subject; and exposing a target site of the subject to an alternating electric field for a period of time, the alternating electric field having a frequency and field strength, wherein the alternating electric field triggers the release of the drug from the drug-loaded nanoparticle at the target site of the subject, wherein the target site comprises cancer cells, wherein the drug kills the cancer cells.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A and FIG. 1B show a schematic representation of A) Self-assembling cationic-anionic polymer nanoparticles (S-CAP NPs) and drug release mechanism; and B) selective targeting mechanism of tumor treating fields triggered targeting of nanoparticles in cancer (TTFields-TTONIC). EPR effect: enhanced permeability and retention effect.

FIG. 2A and FIG. 2B show the electrophoretic device assembly (FIG. 2A); and in vitro drug release from GEM-S-CAP NPs in presence and absence of electric field (FIG. 2B).

FIG. 3 shows chitosan S-CAP NPs were produced with different stabilizer concentration and polymer concentration and assessed by the particle size (in nm), Polydispersity index (how uniform the particle size is in the solution) and percentage of the amount of Gem was encapsulated into these nanoparticles presented by EE (%). For FIG. 3 and FIG. 6 the nanoparticles were formulated to encapsulate Gemcitabine (GEM) in a self-assembling cationic-anionic polymer (S-CAP NPs). Two different polymers were tested-chitosan-bovine serum albumin (BSA) or polyethylenimine (PEI). Nanoparticles were produced by quality by design assisted solvent emulsification evaporation method. Particle size and encapsulation efficacy (EE) were examined.

FIGS. 4A and 4B show a surface response curve representing impact of polymer and stabilizer concentration on (FIG. 4A) particle size (FIG. 4B) encapsulation efficiency of chitosan-bovine serum albumin (BSA) S-CAP NPs. For FIG. 4A, p-value<0.05, particle size increased with polymer concentration (optimum: low polymer concentration), and the stabilizer concentration had inverse correlation with particle size and the effect was non-significant over the concentration of 6%. For FIG. 4B, p-value<0.0001 and both the stabilizer concentration and polymer concentration had direct correlation with EE.

FIG. 5 shows characterization of GEM loaded chitosan-BSA S-CAP NPs. Rows C4 and C7 show the chitosan formulations that were chosen for further analysis. These formulations present high EE while maintaining a relatively small particle size and a low polydispersity index

FIG. 6 polyethylenimine (PEI) S-CAP NPs were produced with different stabilizer concentration and polymer concentration and assessed by the particle size (in nm), Polydispersity index (how uniform the particle size is in the solution) and percentage of the amount of Gem was encapsulated into these nanoparticles presented by EE (%).

FIGS. 7A and 7B show a surface response curve representing impact of polymer and stabilizer concentration on (FIG. 7A) particle size (FIG. 7B) encapsulation efficiency of polyethylenimine (PEI)-BSA S-CAP NPs. For FIG. 7A, p-value<0.0001, polymer concentration had greater impact on particle size and the size increased with increase in polymer concentration. Hence, low polymer concentration (4%) was used in optimized batches. For FIG. 7B, p-value<0.0001 and both the stabilizer concentration and polymer concentration had direct correlation with EE.

FIG. 8 shows characterization of GEM loaded polyethylenimine (PEI)-BSA S-CAP NPs. Rows P5 and P8 show the PEI formulations that were chosen for further analysis. These formulations present high EE while maintaining a relatively small particle size and a low polydispersity index.

FIG. 9 shows a physiochemical characterization of selected S-CAP NPs. In addition to Particle size, Polydispersity index and EE there is also Zeta potential which is the charge of the particle in mV and the percentage of drug content in the particle

FIG. 10 shows in vitro drug release studies. Initial burst release: unencapsulated drug. Sustained drug release profile is very advantageous for controlled drug delivery. Targeted drug release can be achieved by destabilizing the S-CAP NPS under TTFields only in the pancreatic cancer region. Slower drug release from chitosan S-CAP NPs compared to PEI S-CAP NPs exhibit more controlled and sustained drug release.

FIG. 11 shows the stability of chitosan formulations after 1, 3 or 6 months. There are no dramatic changes in particle size, might be some change in polydispersity index (PDI) and in zeta potential but the drug content and 24 hour cumulative drug release is not altered by these changes showing that these formulation are stable following 6 months.

FIG. 12 shows the stability of PEI formulations after 1, 3 or 6 months. There are changes in particle size and as a result in polydispersity index (PDI) and in zeta potential but the drug content and 24 hour cumulative drug release is not altered by these changes showing that these formulation are stable following 6 months but slightly less stable than the chitosan formulations.

FIG. 13 is a physicochemical characterization of selected S-CAP NPs. Significant reduction in EE confirms the TTFields triggered drug release. TTFields destabilize S-Cap-NPs which triggers drug release. EE is the encapsulation efficacy and is calculated by dividing the amount of drug inside the nanoparticle (We) to the amount of drug initially taken to produce the nanoparticles (Wt). Therefore, if TTFields initiate drug release the EE will be lower once TTFields is induced.

FIG. 14 shows a clonogenic-colony formation assay. Significant efficacy with the S-CAP-NPs compared to GEM in a free form. Furthermore, PEI-BSA S-CAP NPs show an increased efficacy (min P<0.01) compared to chitosan-BSA S-CAP NPs.

FIG. 15 shows an inhibition assay. TTFields were 150 KHz. More than an additive effect was observed.

FIG. 16 shows the effect of TTFields on encapsulation efficiency of S-Cap-NPs. Data presented as mean±SD, n=3. Statistical significance was analyzed by one-way ANOVA followed by Dunnett's multiple comparisons test (compared against individual control *P<0.05, **p<0.01).

FIG. 17 shows a cell inhibition assay using Panc—1 cell line. Data presented as mean±SD, n=3. Statistical significance was analyzed by ANOVA followed by Tukey's multiple comparisons test (compared against individual control group ‘TTFields 150 KHz (***P<0.001, ****<0.0001) and S-Cap NP formulation (###P<0.001, ####<0.0001).

FIG. 18 shows a cell inhibition assay using Mia Paca-2 cell line. Data presented as mean±SD, n=3. Statistical significance was analyzed by ANOVA followed by Tukey's multiple comparisons test (compared against individual control group ‘TTFields 150 KHz (***P<0.001, ****<0.0001) and S-Cap NP formulation (###P<0.001, #### <0.0001).

FIG. 19 shows the effect of TTFields on the drug release profile of S-Cap-NPs. Data presented as mean±SD, n=3. Statistical significance was analyzed by multiple t-test for each formulation (*P<0.05, **<0.01).

### DETAILED DESCRIPTION

The disclosed methods and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed, that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a nanoparticle” includes a plurality of such nanoparticles, reference to “the drug” is a reference to one or more drugs and equivalents thereof known to those skilled in the art, and so forth.

As used herein, a “target site” is a specific site or location within or present on a subject or patient. For example, a “target site” can refer to, but is not limited to a cell (e.g. a cancer cell), population of cells, organ, tissue, or a tumor. Thus, the phrase “target cell” can be used to refer to target site, wherein the target site is a cell. In some aspects, organs that can be target sites include, but are not limited to, lung, brain, pancreas, abdominal organs (e.g. stomach, intestine), ovary, breast, uterus, prostate, bladder, liver, colon, or kidney. In some aspects, a cell or population of cells that can be a target site include, but are not limited to, lung cells, brain cells, pancreatic cells, abdominal cells, ovarian cells, liver cells, colon cells, skin cells, or kidney cells. In some aspects, a “target site” can be a tumor target site. In some aspects, a “target site” can be fibrotic tissue or lymph nodes. In some aspects, a “target site” can be a virus-infected cell. In some aspects, a “target cell” can be a cancer. In some aspects, a “target cell” can be a virus-infected cell.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTFields”. TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g. an animal. Thus, the subject of the disclosed methods can be a vertebrate, such as a mammal. For example, the subject can be a human. The term does not denote a particular age or sex. A subject can be used interchangeably with “individual” or “patient.” For example, the subject of administration or exposure to can mean the recipient of the alternating electrical field.

By “treat” is meant to administer or apply a therapeutic, such as alternating electric fields and a vector, to a subject, such as a human or other mammal (for example, an animal model), that has an infection (e.g. viral) or disease (e.g. cancer) or has an increased susceptibility for developing an infection or disease, in order to prevent or delay a worsening of the effects of the disease or infection, or to partially or fully reverse the effects of the infection or disease. For example, treating a subject having cancer can comprise delivering a therapeutic to a cell in the subject.

By “prevent” is meant to minimize or decrease the chance that a subject develops an infection or disease.

As used herein, the terms “administering” and “administration” refer to any method of providing a therapeutic, such as an antiviral agent or anti-cancer therapeutic, to a target site or subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

As used herein, a “therapeutically effective amount” is an amount of a composition (e.g. nanoparticle comprising a therapeutic agent), that provides a therapeutic benefit to an individual or subject. For example, a therapeutically effective amount of a nanoparticle comprising a therapeutic agent is an amount that treats, alleviates, ameliorates, relieves, alleviates symptoms of, prevents, delays onset of, inhibits progression of, reduces severity of, and/or reduces incidence of a disease or infection. In one embodiment, a therapeutically effective amount of a nanoparticle comprising a therapeutic agent will result in an improvement to, or prevents or slows the worsening of, one or more indicators or symptoms of an infection or disease, such as those described herein. As used herein, “treating” a subject with cancer includes administering a therapeutically effective amount of a composition disclosed herein.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

Disclosed are nanoparticles comprising a therapeutic agent. Disclosed are nanoparticles that can be loaded with a compound or drug of interest. Thus, disclosed are drug-loaded nanoparticles. In some aspects, the term “drug” can be used to mean a chemical compound, a peptide, a nucleic acid sequence, an antibody. In some aspects, the term “drug” refers to a therapeutic agent.

In some aspects, the nanoparticle is a cationic-anionic polymer nanoparticle. In some aspects, the nanoparticle is a self-assembling cationic-anionic polymer nanoparticle (S-CAP NP).

In some aspects, the cationic polymer is chitosan or polyethylenimine (PEI). In some aspects, the anionic polymer is bovine serum albumin (BSA).

In some aspects, the drug in the drug-loaded nanoparticles can be, but is not limited to, an anti-cancer drug or cancer therapeutic. In some aspects, the anti-cancer drug can be, but is not limited to, alkylating agents, antimetabolites, natural products, hormones, as well as a variety of other chemicals that do not fall within these discrete classes but are capable of preventing the replication of cancer cells or killing cancer cells. For example, the drug can be, but is not limited to, Gemcitabine (GEM), abraxane, erlotinib, everolimus. In some aspects, the drug can be any therapeutic agent.

In some aspects, the drug in the drug-loaded nanoparticles can be, but is not limited to, a nanoparticle comprising an anti-viral, anti-fungal, anti-bacterial, or any pathogen specific drug.

In some aspects, the drug in the drug-loaded nanoparticles can be, but is not limited to, a nanoparticle comprising a pro-inflammatory molecule or an anti-inflammatory molecule.

### 1. Pharmaceutical Compositions and Delivery

Disclosed herein are pharmaceutical compositions comprising one or more of the nanoparticles described herein. In some aspects, the nanoparticles described herein can be provided in a pharmaceutical composition. For example, the nanoparticles described herein can be formulated with a pharmaceutically acceptable carrier.

In some aspects, a pharmaceutical composition can comprise a therapeutic agent. In some aspects, a pharmaceutical composition can comprise a therapeutic agent and one or more of the nanoparticles described herein. For example, disclosed herein are pharmaceutical compositions comprising one or more of the nanoparticles described herein and a therapeutic such as an anti-cancer drug. In particular, disclosed herein are pharmaceutical compositions comprising one or more of the nanoparticles disclosed herein wherein the nanoparticles are drug-loaded nanoparticles. In some aspects, the drug can be an anti-cancer drug. In some aspects, the anti-cancer drug can be a chemotherapeutic. In some aspects, the chemotherapeutic can be, but is not limited to, an anticancer drug, a cytotoxic drug, pain-management drug, pseudomonas exotoxin A, a non-radioactive isotope (e.g. boron-10 for boron neutron capture therapy), and/or a photosensitizer (e.g. photofrin, foscan, 5-aminolevulinic acid, Mono-L-aspartyl chlorine, pthalocyanines, Meta-tetra(hydroxyphenyl)porphyrins, texaphyrins, Tin ethyl etipurpurin). In some aspects, a chemotherapeutic agent can be, but is not limited to, an alkylating agent, an antimetabolite agent, an antineoplastic antibiotic agent, a mitotic inhibitor agent, a checkpoint inhibitor, and a kinase inhibitor. In some aspects, the drug in the drug-loaded nanoparticles can be, but is not limited to, a nanoparticle comprising an anti-viral, anti-fungal, anti-bacterial, or any pathogen specific drug. In some aspects, the drug in the drug-loaded nanoparticles can be, but is not limited to, a nanoparticle comprising a pro-inflammatory molecule or an anti-inflammatory molecule.

Any of the therapeutic agents listed above as examples of a sequence of interest can also be provided separate from the vector as part of the pharmaceutical composition.

Disclosed herein are compositions comprising one or more of the nanoparticles described herein that further comprise a carrier such as a pharmaceutically acceptable carrier. For example, disclosed are pharmaceutical compositions, comprising the nanoparticles disclosed herein, and a pharmaceutically acceptable carrier.

For example, pharmaceutical compositions comprising the nanoparticles described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidylcholine (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995, which is hereby incorporated by reference in its entirety for the teaching of other suitable pharmaceutically acceptable carriers. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of vector being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

In the methods described herein, delivery (or administration or introduction) of the vector or pharmaceutical compositions disclosed herein to subjects can be via a variety of mechanisms.

Disclosed are methods comprising introducing a nanoparticle to a cell. In some aspects, the methods comprising introducing a nanoparticle to a target site. In some aspects the methods comprise introducing a nanoparticle to a subject. Thus, a subject comprises a cell that is the target site for the disclosed nanoparticles. In some aspects, all of the disclosed methods comprising introducing a nanoparticle to a cell also comprise introducing a nanoparticle to a subject.

### C. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body.

In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### D. Methods of Delivering a Therapeutic to a Target Site

Disclosed are methods of delivering a therapeutic to a target site of a subject comprising administering a nanoparticle to a target site of a subject, wherein the nanoparticle comprises a therapeutic agent; and applying an alternating electric field, at a frequency for a period of time, to the target site of the subject, wherein the alternating electric field releases the therapeutic from the nanoparticle at the target site of the subject.

In some aspects, the nanoparticle is loaded with a therapeutic agent. In some aspects, the therapeutic agent is encapsulated in the nanoparticle. Thus, the nanoparticles can be actively or passively loaded with a therapeutic agent. In some aspects, the nanoparticle is coated with a therapeutic agent. In some aspects, the therapeutic agent is conjugated to the nanoparticle. For example, the conjugation can be via a linker. In some aspects, the linker can be cleavable. There are several ways a nanoparticle can “carry” a therapeutic agent, all of which are considered herein.

In some aspects, the therapeutic agent can be a small molecule, nucleic acid, carbohydrate, lipid, peptide, antibody, or antibody fragment. In some aspects, the therapeutic agent can be any composition capable of treating a disease of interest. For example, a therapeutic agent can be, but is not limited to, an anti-cancer therapeutic, an anti-viral therapeutic, an anti-bacterial therapeutic, an anti-fungal therapeutic, a pro-inflammatory therapeutic, or an anti-inflammatory therapeutic. In some aspects, an anti-cancer therapeutic can be Gemcitabine (GEM), paclitaxel, doxorubicin, cytarabine, daunorubicin, or any chemotherapeutic. In some aspects, a therapeutic agent can be a nucleic acid, siRNA, an antisense oligonucleotide, or an aptamer.

In some aspects, the nanoparticle comprises a cell-specific targeting moiety. In some aspects, the cell-specific targeting moiety can be a cancer cell-specific targeting moiety.

In some aspects, the targeting moiety can direct, or target, the nanoparticle to a specific cell. The cell-specific targeting moiety can be a chemical, compound, peptide or nucleic acid. Examples of targeting moieties include, but are not limited to, molecules that recognize receptors on specific cell types.

In some aspects, the nanoparticle can comprise a cell penetrating peptide. In some aspects, a cell penetrating peptide can facilitate the delivery of the nanoparticle to the cytoplasm of the cell.

In some aspects, the nanoparticle is labeled. As used herein, a label, or detection agent, is any molecule that can be associated with a nanoparticle, directly or indirectly, and which results in a measurable, detectable signal, either directly or indirectly. Many such labels for incorporation into nanoparticles or coupling to nanoparticles are known to those of skill in the art. Examples of detection agents can be, but are not limited to, radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluorophores are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase.

In some aspects, the nanoparticle is a cationic-anionic polymer nanoparticle. The cationic-anionic make up of a nanoparticle allows for alternating electric fields to cause the high charge density cationic and anionic polymers to experience alignment and migration based on their charge, thus leading to disruption (or bursting) of the nanoparticle. In some aspects, the nanoparticle is a self-assembling cationic-anionic polymer nanoparticle. In some aspects, the cationic polymer can be chitosan or polyethylenimine (PEI), Poly (2-N,N-dimethylaminoethylmethacrylate-PDMAEMA), Poly (amidoamine) (PAMAM), polylysine-based polyplexes, Natural cationic polymers, cationic gelatin, cationic cellulose, cationic dextran. In some aspects, the anionic polymer can be bovine serum albumin (BSA), Polyacrylic acid cystamine conjugate, CMS-sodium carboxy methyl starch, CMG carboxy methyl guar gum, carboxymethyl cellulose, Sodium alginate.

In some aspects, the nanoparticle is 20-500 nm. In some aspects, the nanoparticle is 50-400 nm. In some aspects, the nanoparticle is 50-150 nm. In some aspects, the nanoparticle is 100-300 nm.

In some aspects, the target site comprises a cell. For example, the cell can be, but is not limited to, a cancer cell, a pathogen-infected cell, a mutant cell, or an immune cell. In some aspects, a cancer cell can be, but is not limited to, a pancreatic cancer cell, glioblastoma cell, colon cancer cell, skin cancer cell, or lung metastatic carcinoma cell. In some aspects, a pathogen-infected cell can be a virus infected, bacteria infected, or fungus infected cell. In some aspects, a mutant cell can be a benign tumor cell, such as a benign topical tumor cell. Thus, a mutant cell can be a cell that may not be a disease-specific cell but also is not a healthy, wild type cell. In some aspects, the nanoparticle enters the target site. For example, in some aspects, target site is a cancer cell or pathogen-infected cell and the nanoparticle can enter the cancer cell or pathogen-infected cell. In some aspects, the target site is the lungs and the nanoparticle can enter the lungs.

In some aspects, the subject has cancer, an infection, an inflammatory disorder, or is immune suppressed. Thus, in some aspects, the therapeutic is an anti-cancer, anti-infectious agent, anti-inflammatory or pro-inflammatory therapeutic.

In some aspects, the frequency of the alternating electric field is between 100 and 1,000 kHz. In some aspects, the frequency of the alternating electric field is between 100 and 500 kHz. In some aspects, the frequency of the alternating electric field is between 150 and 300 kHz. In some aspects, the frequency of the alternating electric field is between 180 and 220 kHz.

Also disclosed are methods of delivering a therapeutic to a target site of a subject comprising administering a nanoparticle to a target site of a subject, wherein the nanoparticle comprises a therapeutic agent; and applying an alternating electric field, at a frequency for a period of time, to the target site of the subject, wherein the alternating electric field releases the therapeutic from nanoparticle at the target site of the subject, and further comprising applying a second alternating electric field at a second frequency to the target site prior to, during or after the step of administering the nanoparticle to the target site of the subject. Thus, disclosed are methods that allow the nanoparticle to get to a target site and the alternating electric fields cause the nanoparticle to burst thus releasing the therapeutic from the nanoparticle near a cell but not within the cell. Also disclosed are methods that allow the nanoparticle to get to a target site and a first alternating electric field causes the nanoparticle to enter a cell and a second alternating electric field causes the nanoparticle to burst thus releasing the therapeutic from the nanoparticle within the cell. In some aspects, the second alternating electric field increases permeability of a cell membrane of a cell at the target site of the subject.

In some aspects, when a second alternating electric field is applied, the first and second alternating electric fields can be at the same frequency or at different frequencies. In some aspects, the first and second alternating electric fields can be at different lengths of time or for the same length of time.

In some aspects, the nanoparticle is administered prior to exposing the target site to the alternating electric field. In some aspects, the nanoparticle is administered at the same time as the alternating electric field. In some aspects, the nanoparticle is administered after the alternating electric field.

### E. Methods of Increasing Target Site Specific Release of a Drug

Disclosed are methods of increasing target site specific release of a therapeutic agent in a subject comprising administering a nanoparticle to a target site of a subject, wherein the nanoparticle comprises a therapeutic agent; and applying an alternating electric field, at a frequency for a period of time, to the target site of the subject, wherein the alternating electric field releases the therapeutic agent from the nanoparticle at the target site of the subject, thereby increasing the target site specific release of the therapeutic agent.

In some aspects, an increase in the target site specific release of the therapeutic agent can be determined by comparing the release of the therapeutic agent at the target site in the presence and absence of alternating electric fields. An increase in the target site specific release of the therapeutic agent can be detected in the presence of alternating electric fields compared to in the absence of alternating electric fields.

In some aspects, the nanoparticle is loaded with a therapeutic agent. In some aspects, the therapeutic agent is encapsulated in the nanoparticle. Thus, the nanoparticles can be actively or passively loaded with a therapeutic agent. In some aspects, the nanoparticle is coated with a therapeutic agent. In some aspects, the therapeutic agent is conjugated to the nanoparticle. For example, the conjugation can be via a linker. In some aspects, the linker can be cleavable. There are several ways a nanoparticle can “carry” a therapeutic agent, all of which are considered herein.

In some aspects, the therapeutic agent can be a small molecule, nucleic acid, carbohydrate, lipid, peptide, antibody, or antibody fragment. In some aspects, the therapeutic agent can be any composition capable of treating a disease of interest. For example, a therapeutic agent can be, but is not limited to, an anti-cancer therapeutic, an anti-viral therapeutic, an anti-bacterial therapeutic, an anti-fungal therapeutic, a pro-inflammatory therapeutic, or an anti-inflammatory therapeutic. In some aspects, an anti-cancer therapeutic can be Gemcitabine (GEM), paclitaxel, doxorubicin, cytarabine, daunorubicin, or any chemotherapeutic. In some aspects, a therapeutic agent can be a siRNA, antisense oligonucleotide, or aptamers.

In some aspects, the nanoparticle can comprise a cell-specific targeting moiety. In some aspects, the cell-specific targeting moiety can be a cancer cell-specific targeting moiety.

In some aspects, the targeting moiety can direct, or target, the nanoparticle to a specific cell or group of cells. The cell-specific targeting moiety can be a chemical, compound, peptide or nucleic acid. Examples of targeting moieties include, but art not limited to, molecules that recognize receptors on specific cell types.

In some aspects, the nanoparticle can comprise a cell penetrating peptide. In some aspects, a cell penetrating peptide can facilitate the delivery of the nanoparticle to the cytoplasm of the cell.

In some aspects, the nanoparticle is labeled. As used herein, a label, or detection agent, is any molecule that can be associated with a nanoparticle, directly or indirectly, and which results in a measurable, detectable signal, either directly or indirectly. Many such labels for incorporation into nanoparticles or coupling to nanoparticles are known to those of skill in the art. Examples of detection agents can be, but are not limited to, radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase.

In some aspects, the nanoparticle is a cationic-anionic polymer nanoparticle. The cationic-anionic make up of a nanoparticle allows for alternating electric fields to cause the high charge density cationic and anionic polymers to experience alignment and migration based on their charge, thus leading to disruption (or bursting) of the nanoparticle. In some aspects, the nanoparticle is a self-assembling cationic-anionic polymer nanoparticle. In some aspects, the cationic polymer can be chitosan or polyethylenimine (PEI), Poly (2-N,N-dimethylaminoethylmethacrylate-PDMAEMA), Poly (amidoamine) (PAMAM), polylysine-based polyplexes, Natural cationic polymers, cationic gelatin, cationic cellulose, cationic dextran. In some aspects, the anionic polymer can be bovine serum albumin (BSA), Polyacrylic acid cystamine conjugate, CMS-sodium carboxy methyl starch, CMG carboxy methyl guar gum, carboxymethyl cellulose, Sodium alginate.

In some aspects, the nanoparticle is 20-500 nm. In some aspects, the nanoparticle is 50-400 nm. In some aspects, the nanoparticle is 50-150 nm. In some aspects, the nanoparticle is 100-300 nm.

In some aspects, the target site comprises a cell. For example, the cell can be, but is not limited to, a cancer cell, a pathogen-infected cell, a mutant cell, or an immune cell. In some aspects, a cancer cell can be, but is not limited to, a pancreatic cancer cell, glioblastoma cell, colon cancer cell, skin cancer cell, or lung metastatic carcinoma cell. In some aspects, a pathogen-infected cell can be a virus infected, bacteria infected, or fungus infected cell. In some aspects, a mutant cell can be a benign tumor cell, such as a benign topical tumor cell. Thus, a mutant cell can be a cell that may not be a disease-specific cell but also is not a healthy, wild type cell. In some aspects, the nanoparticle enters the target site. For example, in some aspects, target site is a cancer cell or pathogen-infected cell and the nanoparticle can enter the cancer cell or pathogen-infected cell. In some aspects, the target site is the lungs and the nanoparticle can enter the lungs.

In some aspects, the subject has cancer, an infection, an inflammatory disorder, or is immune suppressed. Thus, in some aspects, the therapeutic is an anti-cancer, anti-infectious agent, anti-inflammatory or pro-inflammatory therapeutic.

In some aspects, the frequency of the alternating electric field is between 100 and 1,000 kHz. In some aspects, the frequency of the alternating electric field is between 100 and 500 kHz. In some aspects, the frequency of the alternating electric field is between 150 and 300 kHz. In some aspects, the frequency of the alternating electric field is between 180 and 220 kHz.

Also disclosed are methods of increasing target site specific release of a therapeutic to a target site of a subject comprising administering a nanoparticle to a target site of a subject, wherein the nanoparticle comprises a therapeutic agent; and applying an alternating electric field, at a frequency for a period of time, to the target site of the subject, wherein the alternating electric field releases the therapeutic from nanoparticle at the target site of the subject, thereby increasing the target site specific release of the therapeutic agent and further comprising applying a second alternating electric field at a second frequency to the target site prior to, during or after the step of administering the nanoparticle to the target site of the subject. Thus, disclosed are methods that allow the nanoparticle to get to a target site and the alternating electric fields cause the nanoparticle to burst, thus releasing the therapeutic from the nanoparticle near a cell but not within the cell. Also disclosed are methods that allow the nanoparticle to get to a target site and a first alternating electric field causes the nanoparticle to enter a cell and a second alternating electric field causes the nanoparticle to burst, thereby releasing the therapeutic from the nanoparticle within the cell. In some aspects, the second alternating electric field increases permeability of a cell membrane of a cell at the target site of the subject.

In some aspects, when a second alternating electric field is applied, the first and second alternating electric fields can be at the same frequency or at different frequencies. In some aspects, the first and second alternating electric fields can be at different lengths of time or for the same length of time.

In some aspects, the nanoparticle is administered prior to exposing the target site to the alternating electric field. In some aspects, the nanoparticle is administered at the same time as the alternating electric field. In some aspects, the nanoparticle is administered after the alternating electric field.

### F. Methods of Treating

Disclosed are methods of treating a subject in need thereof, wherein the subject in need thereof has cancer, has an infection, has an inflammatory disorder, or is immune suppressed. In some aspects, the methods of treating comprise administering a nanoparticle to a target site of a subject in need thereof, wherein the nanoparticle comprises a therapeutic agent; and applying an alternating electric field, at a frequency for a period of time, to the target site of the subject in need thereof, wherein the alternating electric field releases the therapeutic agent from the nanoparticle at the target site of the subject in need thereof. Once released, the drug can provide a therapeutic effect. In some aspects, the therapeutic effect is to kill the cell (such as a cancer cell or a pathogen infected cell), reduce inflammation, or increase the humoral and/or cell-mediated immune response.

In some aspects, the subject has cancer, an infection, an inflammatory disorder, or is immune suppressed. In some aspects, the subject has a neurodegenerative disease, autoimmune disease, or orthopedic condition. In some aspects, the infection can be a viral, bacterial or fungal infection. In some aspects, the cancer can be brain cancer, pancreatic cancer, lung cancer, ovarian cancer, colon cancer, skin cancer, or breast cancer. Thus, in some aspects, the therapeutic is an anti-cancer, anti-infectious agent, anti-inflammatory or pro-inflammatory therapeutic.

Disclosed are methods of treating cancer in a subject comprising administering a nanoparticle to a target site of a subject having cancer, wherein the nanoparticle comprises a therapeutic agent; and applying an alternating electric field, at a frequency for a period of time, to the target site of the subject having cancer, wherein the target site comprises a cancer cell; wherein the alternating electric field releases the therapeutic agent from the nanoparticle at the target site of the subject; wherein the therapeutic kills the cancer cell.

In some aspects, the nanoparticle is loaded with a therapeutic agent. In some aspects, the therapeutic agent is encapsulated in the nanoparticle. Thus, the nanoparticle can be actively or passively loaded with a therapeutic agent. In some aspects, the nanoparticle is coated with a therapeutic agent. In some aspects, the therapeutic agent is conjugated to the nanoparticle. For example, the conjugation can be via a linker. In some aspects, the linker can be cleavable. There are several ways a nanoparticle can “carry” a therapeutic agent, all of which are considered herein.

In some aspects, the therapeutic agent can be a small molecule, nucleic acid, carbohydrate, lipid, peptide, antibody, or antibody fragment. In some aspects, the therapeutic agent can be any composition capable of treating a disease of interest. For example, a therapeutic agent can be, but is not limited to, an anti-cancer therapeutic, an anti-viral therapeutic, an anti-bacterial therapeutic, an anti-fungal therapeutic, a pro-inflammatory therapeutic, or an anti-inflammatory therapeutic. In some aspects, an anti-cancer therapeutic can be Gemcitabine (GEM), paclitaxel, doxorubicin, cytarabine, daunorubicin, or any chemotherapeutic. In some aspects, a therapeutic agent can be a siRNA, antisense oligonucleotide, or aptamers.

In some aspects, the nanoparticle comprises a cell-specific targeting moiety. In some aspects, the cell-specific targeting moiety can be a cancer cell-specific targeting moiety.

In some aspects, the targeting moiety can direct, or target, the nanoparticle to a specific cell. The cell-specific targeting moiety can be a chemical, compound, peptide or nucleic acid. Examples of targeting moieties include, but are not limited to, molecules that recognize receptors on specific cell types.

In some aspects, the nanoparticle can comprise a cell penetrating peptide. In some aspects, a cell penetrating peptide can facilitate the delivery of the nanoparticle to the cytoplasm of the cell.

In some aspects, the nanoparticle is labeled. As used herein, a label, or detection agent, is any molecule that can be associated with a nanoparticle, directly or indirectly, and which results in a measurable, detectable signal, either directly or indirectly. Many such labels for incorporation into nanoparticles or coupling to nanoparticles are known to those of skill in the art. Examples of detection agents can be, but are not limited to, radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluorophores are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase.

In some aspects, the nanoparticle is a cationic-anionic polymer nanoparticle. The cationic-anionic make up of a nanoparticle allows for alternating electric fields to cause the high charge density cationic and anionic polymers to experience alignment and migration based on their charge, thus leading to disruption (or bursting) of the nanoparticle. In some aspects, the nanoparticle is a self-assembling cationic-anionic polymer nanoparticle. In some aspects, the cationic polymer can be chitosan or polyethylenimine (PEI), Poly (2-N,N-dimethylaminoethylmethacrylate-PDMAEMA), Poly (amidoamine) (PAMAM), polylysine-based polyplexes, Natural cationic polymers, cationic gelatin, cationic cellulose, cationic dextran. In some aspects, the anionic polymer can be bovine serum albumin (BSA), Polyacrylic acid cystamine conjugate, CMS-sodium carboxy methyl starch, CMG carboxy methyl guar gum, carboxymethyl cellulose, Sodium alginate.

In some aspects, the nanoparticle is 20-500 nm. In some aspects, the nanoparticle is 50-400 nm. In some aspects, the nanoparticle is 50-150 nm. In some aspects, the nanoparticle is 100-300 nm.

In some aspects, the target site comprises a cell. For example, the cell can be, but is not limited to, a cancer cell, a pathogen-infected cell, a mutant cell, or an immune cell. In some aspects, a cancer cell can be, but is not limited to, a pancreatic cancer cell, glioblastoma cell, colon cancer cell, skin cancer cell, or lung metastatic carcinoma cell. In some aspects, a pathogen-infected cell can be a virus infected, bacteria infected, or fungus infected cell. In some aspects, a mutant cell can be a benign tumor cell, such as a benign topical tumor cell. Thus, a mutant cell can be a cell that may not be a disease-specific cell but also is not a healthy, wild type cell. In some aspects, the nanoparticle enters the target site. For example, in some aspects, target site is a cancer cell or pathogen-infected cell and the nanoparticle can enter the cancer cell or pathogen-infected cell. In some aspects, the target site is the lungs and the nanoparticle can enter the lungs.

In some aspects, the subject has cancer, an infection, an inflammatory disorder, or is immune suppressed. Thus, in some aspects, the therapeutic is an anti-cancer, anti-infectious agent, anti-inflammatory or pro-inflammatory therapeutic.

In some aspects, the frequency of the alternating electric field is between 100 and 1,000 kHz. In some aspects, the frequency of the alternating electric field is between 100 and 500 kHz. In some aspects, the frequency of the alternating electric field is between 150 and 300 kHz. In some aspects, the frequency of the alternating electric field is between 180 and 220 kHz.

Also disclosed are methods of treating a subject in need thereof comprising administering a nanoparticle to a target site of a subject, wherein the nanoparticle comprises a therapeutic agent; and applying an alternating electric field, at a frequency for a period of time, to the target site of the subject, wherein the alternating electric field releases the therapeutic from nanoparticle at the target site of the subject, and further comprising applying a second alternating electric field at a second frequency to the target site prior to, during or after the step of administering the nanoparticle to the target site of the subject. Thus, disclosed are methods that allow the nanoparticle to get to a target site and the alternating electric fields cause the nanoparticle to burst thus releasing the therapeutic from the nanoparticle near a cell but not within the cell. Also disclosed are methods that allow the nanoparticle to get to a target site and a first alternating electric field causes the nanoparticle to enter a cell and a second alternating electric field causes the nanoparticle to burst thus releasing the therapeutic from the nanoparticle within the cell. In some aspects, the second alternating electric field increases permeability of a cell membrane of a cell at the target site of the subject.

In some aspects, when a second alternating electric field is applied, the first and second alternating electric fields can be at the same frequency or at different frequencies. In some aspects, the first and second alternating electric fields can be at different lengths of time or for the same length of time.

In some aspects, the nanoparticle is administered prior to exposing the target site to the alternating electric field. In some aspects, the nanoparticle is administered at the same time as the alternating electric field. In some aspects, the nanoparticle is administered after the alternating electric field.

### G. Methods of Killing Cells

Disclosed are methods of killing a cell comprising administering a nanoparticle to a target site, wherein the nanoparticle comprises a therapeutic agent; and applying an alternating electric field for a period of time, to the target site, wherein the alternating electric field releases the therapeutic agent from the nanoparticle at the target site, wherein the target site comprises a cell, wherein the therapeutic kills the cell.

In some aspects, the disclosed methods of killing a cell occurs in vivo. Thus, in some aspects, administering a nanoparticle to a target site comprises administering a nanoparticle to a target site of a subject. In some aspects, the disclosed methods of killing a cell occurs in vitro. Thus, in some aspects, administering a nanoparticle to a target site comprises administering a nanoparticle to a culture dish comprising cells.

In some aspects, a cell can be a cancer cell or a pathogen-infected cell. In some aspects, the subject has cancer, an infection, or an inflammatory disorder. In some aspects, the subject has a neurodegenerative disease, autoimmune disease, or orthopedic condition. In some aspects, the infection can be a viral, bacterial or fungal infection. In some aspects, the cancer can be brain cancer, pancreatic cancer, lung cancer, ovarian cancer, colon cancer, skin cancer, or breast cancer. Thus, in some aspects, the therapeutic is an anti-cancer, anti-infectious agent, or anti-inflammatory.

In some aspects, the nanoparticle is loaded with a therapeutic agent. In some aspects, the therapeutic agent is encapsulated in the nanoparticle. Thus, the nanoparticles can be actively or passively loaded with a therapeutic agent. In some aspects, the nanoparticle is coated with a therapeutic agent. In some aspects, the therapeutic agent is conjugated to the nanoparticle. For example, the conjugation can be via a linker. In some aspects, the linker can be cleavable. There are several ways a nanoparticle can “carry” a therapeutic agent, all of which are considered herein.

In some aspects, the therapeutic agent can be a small molecule, nucleic acid, carbohydrate, lipid, peptide, antibody, or antibody fragment. In some aspects, the therapeutic agent can be any composition capable of treating a disease of interest. For example, a therapeutic agent can be, but is not limited to, an anti-cancer therapeutic, an anti-viral therapeutic, an anti-bacterial therapeutic, an anti-fungal therapeutic, a pro-inflammatory therapeutic, or an anti-inflammatory therapeutic. In some aspects, an anti-cancer therapeutic can be Gemcitabine (GEM), paclitaxel, doxorubicin, cytarabine, daunorubicin, or any chemotherapeutic. In some aspects, a therapeutic agent can be a siRNA, antisense oligonucleotide, or aptamers.

In some aspects, the nanoparticle comprises a cell-specific targeting moiety. In some aspects, the cell-specific targeting moiety can be a cancer cell-specific targeting moiety.

In some aspects, the targeting moiety can direct, or target, the nanoparticle to a specific cell. The cell-specific targeting moiety can be a chemical, compound, peptide or nucleic acid. Examples of targeting moieties include, but art not limited to, molecules that recognize receptors on specific cell types.

In some aspects, the nanoparticle can comprise a cell penetrating peptide. In some aspects, a cell penetrating peptide can facilitate the delivery of the nanoparticle to the cytoplasm of the cell.

In some aspects, the nanoparticle is labeled. As used herein, a label, or detection agent, is any molecule that can be associated with a nanoparticle, directly or indirectly, and which results in a measurable, detectable signal, either directly or indirectly. Many such labels for incorporation into nanoparticles or coupling to nanoparticles are known to those of skill in the art. Examples of detection agents can be, but are not limited to, radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluorophores are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase.

In some aspects, the nanoparticle is a cationic-anionic polymer nanoparticle. The cationic-anionic make up of a nanoparticle allows for alternating electric fields to cause the high charge density cationic and anionic polymers to experience alignment and migration based on their charge, thus leading to disruption (or bursting) of the nanoparticle. In some aspects, the nanoparticle is a self-assembling cationic-anionic polymer nanoparticle. In some aspects, the cationic polymer can be chitosan or polyethylenimine (PEI), Poly (2-N,N-dimethylaminoethylmethacrylate-PDMAEMA), Poly (amidoamine) (PAMAM), polylysine-based polyplexes, Natural cationic polymers, cationic gelatin, cationic cellulose, cationic dextran. In some aspects, the anionic polymer can be bovine serum albumin (BSA), Polyacrylic acid cystamine conjugate, CMS-sodium carboxy methyl starch, CMG carboxy methyl guar gum, carboxymethyl cellulose, Sodium alginate.

In some aspects, the nanoparticle is 20-500 nm. In some aspects, the nanoparticle is 50-400 nm. In some aspects, the nanoparticle is 50-150 nm. In some aspects, the nanoparticle is 100-300 nm.

In some aspects, the target site comprises a cell. For example, the cell can be, but is not limited to, a cancer cell, a pathogen-infected cell, a mutant cell, or an immune cell. In some aspects, a cancer cell can be, but is not limited to, a pancreatic cancer cell, glioblastoma cell, colon cancer cell, skin cancer cell, or lung metastatic carcinoma cell. In some aspects, a pathogen-infected cell can be a virus infected, bacteria infected, or fungus infected cell. In some aspects, a mutant cell can be a benign tumor cell, such as a benign topical tumor cell. Thus, a mutant cell can be a cell that may not be a disease-specific cell but also is not a healthy, wild type cell. In some aspects, the nanoparticle enters the target site. For example, in some aspects, target site is a cancer cell or pathogen-infected cell and the nanoparticle can enter the cancer cell or pathogen-infected cell. In some aspects, the target site is the lungs and the nanoparticle can enter the lungs. In some aspects, the target site can comprise fibrotic tissue or cancer tissue. In some aspects, the target site can be a joint.

In some aspects, the subject has cancer, an infection, an inflammatory disorder, or is immune suppressed. Thus, in some aspects, the therapeutic is an anti-cancer, anti-infectious agent, anti-inflammatory or pro-inflammatory therapeutic.

In some aspects, the frequency of the alternating electric field is between 100 and 1,000 kHz. In some aspects, the frequency of the alternating electric field is between 100 and 500 kHz. In some aspects, the frequency of the alternating electric field is between 150 and 300 kHz. In some aspects, the frequency of the alternating electric field is between 180 and 220 kHz.

Also disclosed are methods of killing a cell comprising administering a nanoparticle to a target site, wherein the nanoparticle comprises a therapeutic agent; and applying an alternating electric field for a period of time, to the target site, wherein the alternating electric field releases the therapeutic agent from the nanoparticle at the target site, wherein the target site comprises a cell, wherein the therapeutic kills the cell, and further comprising applying a second alternating electric field at a second frequency to the target site prior to, during or after the step of administering the nanoparticle to the target site of the subject.

Thus, disclosed are methods that allow the nanoparticle to get to a target site and the alternating electric fields cause the nanoparticle to burst thus releasing the therapeutic from the nanoparticle near a cell but not within the cell. Also disclosed are methods that allow the nanoparticle to get to a target site and a first alternating electric field causes the nanoparticle to enter a cell and a second alternating electric field causes the nanoparticle to burst thus releasing the therapeutic from the nanoparticle within the cell. In some aspects, the second alternating electric field increases permeability of a cell membrane of a cell at the target site of the subject.

In some aspects, when a second alternating electric field is applied, the first and second alternating electric fields can be at the same frequency or at different frequencies. In some aspects, the first and second alternating electric fields can be at different lengths of time or for the same length of time.

In some aspects, the nanoparticle is administered prior to exposing the target site to the alternating electric field. In some aspects, the nanoparticle is administered at the same time as the alternating electric field. In some aspects, the nanoparticle is administered after the alternating electric field.

### H. Methods of Disrupting Nanoparticles

Disclosed are methods of disrupting a nanoparticle comprising administering a nanoparticle to a target site, wherein the nanoparticle comprises a therapeutic agent; and applying an alternating electric field for a period of time, to the target site, wherein the alternating electric field disrupts the nanoparticle and releases the therapeutic agent from the nanoparticle at the target site, wherein the target site comprises a cell. In some aspects, release of the therapeutic kills one or more cells at the target site.

In some aspects, the disclosed methods of disrupting a nanoparticle occur in vivo. Thus, in some aspects, administering a nanoparticle to a target site comprises administering a nanoparticle to a target site of a subject. In some aspects, the disclosed methods of disrupting a nanoparticle occur in vitro. Thus, in some aspects, administering a nanoparticle to a target site comprises administering a nanoparticle to a culture dish comprising cells.

In some aspects, a cell can be a cancer cell or a pathogen-infected cell. In some aspects, the subject has cancer, an infection, or an inflammatory disorder. In some aspects, the subject has a neurodegenerative disease, autoimmune disease, or orthopedic condition. In some aspects, the infection can be a viral, bacterial or fungal infection. In some aspects, the cancer can be brain cancer, pancreatic cancer, lung cancer, ovarian cancer, colon cancer, skin cancer, or breast cancer. Thus, in some aspects, the therapeutic is an anti-cancer, anti-infectious agent, or anti-inflammatory.

In some aspects, the nanoparticle is loaded with a therapeutic agent. In some aspects, the therapeutic agent is encapsulated in the nanoparticle. Thus, the nanoparticles can be actively or passively loaded with a therapeutic agent. In some aspects, the nanoparticle is coated with a therapeutic agent. In some aspects, the therapeutic agent is conjugated to the nanoparticle. For example, the conjugation can be via a linker. In some aspects, the linker can be cleavable. There are several ways a nanoparticle can “carry” a therapeutic agent, all of which are considered herein.

In some aspects, the therapeutic agent can be a small molecule, nucleic acid, carbohydrate, lipid, peptide, antibody, or antibody fragment. In some aspects, the therapeutic agent can be any composition capable of treating a disease of interest. For example, a therapeutic agent can be, but is not limited to, an anti-cancer therapeutic, an anti-viral therapeutic, an anti-bacterial therapeutic, an anti-fungal therapeutic, a pro-inflammatory therapeutic, or an anti-inflammatory therapeutic. In some aspects, an anti-cancer therapeutic can be Gemcitabine (GEM), paclitaxel, doxorubicin, cytarabine, daunorubicin, or any chemotherapeutic. In some aspects, a therapeutic agent can be a siRNA, antisense oligonucleotide, or aptamers.

In some aspects, the nanoparticle comprises a cell-specific targeting moiety. In some aspects, the cell-specific targeting moiety can be a cancer cell-specific targeting moiety.

In some aspects, the targeting moiety can direct, or target, the nanoparticle to a specific cell. The cell-specific targeting moiety can be a chemical, compound, peptide or nucleic acid. Examples of targeting moieties include, but art not limited to, molecules that recognize receptors on specific cell types.

In some aspects, the nanoparticle can comprise a cell penetrating peptide. In some aspects, a cell penetrating peptide can facilitate the delivery of the nanoparticle to the cytoplasm of the cell.

In some aspects, the nanoparticle is labeled. As used herein, a label, or detection agent, is any molecule that can be associated with a nanoparticle, directly or indirectly, and which results in a measurable, detectable signal, either directly or indirectly. Many such labels for incorporation into nanoparticles or coupling to nanoparticles are known to those of skill in the art. Examples of detection agents can be, but are not limited to, radioactive isotopes, fluorescent molecules, phosphorescent molecules, enzymes, antibodies, and ligands. Any of the disclosed detection agents can be used.

In some aspects, the nanoparticle is a cationic-anionic polymer nanoparticle. The cationic-anionic make up of a nanoparticle allows for alternating electric fields to cause the high charge density cationic and anionic polymers to experience alignment and migration based on their charge, thus leading to disruption (or bursting) of the nanoparticle. In some aspects, the nanoparticle is a self-assembling cationic-anionic polymer nanoparticle. In some aspects, the cationic polymer can be chitosan or polyethylenimine (PEI), Poly (2-N,N-dimethylaminoethylmethacrylate-PDMAEMA), Poly (amidoamine) (PAMAM), polylysine-based polyplexes, Natural cationic polymers, cationic gelatin, cationic cellulose, cationic dextran. In some aspects, the anionic polymer can be bovine serum albumin (BSA), Polyacrylic acid cystamine conjugate, CMS-sodium carboxy methyl starch, CMG carboxy methyl guar gum, carboxymethyl cellulose, Sodium alginate.

In some aspects, the nanoparticle is 20-500 nm. In some aspects, the nanoparticle is 50-400 nm. In some aspects, the nanoparticle is 50-150 nm. In some aspects, the nanoparticle is 100-300 nm.

In some aspects, the target site comprises a cell. For example, the cell can be, but is not limited to, a cancer cell, a pathogen-infected cell, a mutant cell, or an immune cell. In some aspects, a cancer cell can be, but is not limited to, a pancreatic cancer cell, glioblastoma cell, colon cancer cell, skin cancer cell, or lung metastatic carcinoma cell. In some aspects, a pathogen-infected cell can be a virus infected, bacteria infected, or fungus infected cell. In some aspects, a mutant cell can be a benign tumor cell, such as a benign topical tumor cell. Thus, a mutant cell can be a cell that may not be a disease-specific cell but also is not a healthy, wild type cell. In some aspects, the nanoparticle enters the target site. For example, in some aspects, target site is a cancer cell or pathogen-infected cell and the nanoparticle can enter the cancer cell or pathogen-infected cell. In some aspects, the target site is the lungs and the nanoparticle can enter the lungs. In some aspects, the target site can comprise fibrotic tissue or cancer tissue. In some aspects, the target site can be a joint.

In some aspects, the subject has cancer, an infection, an inflammatory disorder, or is immune suppressed. Thus, in some aspects, the therapeutic is an anti-cancer, anti-infectious agent, anti-inflammatory or pro-inflammatory therapeutic.

In some aspects, the frequency of the alternating electric field is between 100 and 1,000 kHz. In some aspects, the frequency of the alternating electric field is between 100 and 500 kHz. In some aspects, the frequency of the alternating electric field is between 150 and 300 kHz. In some aspects, the frequency of the alternating electric field is between 180 and 220 kHz.

### I. Drug Delivery System

Disclosed are drug delivery systems comprising a nanoparticle comprising a therapeutic agent; and a device capable of administering an alternating electric field.

In some aspects, the nanoparticle is loaded with a therapeutic agent. In some aspects, the therapeutic agent is encapsulated in the nanoparticle. Thus, the nanoparticles can be actively or passively loaded with a therapeutic agent. In some aspects, the nanoparticle is coated with a therapeutic agent. In some aspects, the therapeutic agent is conjugated to the nanoparticle. For example, the conjugation can be via a linker. In some aspects, the linker can be cleavable. There are several ways a nanoparticle can “carry” a therapeutic agent, all of which are considered herein.

In some aspects, the therapeutic agent can be a small molecule, nucleic acid, carbohydrate, lipid, peptide, antibody, or antibody fragment. In some aspects, the therapeutic agent can be any composition capable of treating a disease of interest. For example, a therapeutic agent can be, but is not limited to, an anti-cancer therapeutic, an anti-viral therapeutic, an anti-bacterial therapeutic, an anti-fungal therapeutic, a pro-inflammatory therapeutic, or an anti-inflammatory therapeutic. In some aspects, an anti-cancer therapeutic can be Gemcitabine (GEM), paclitaxel, doxorubicin, cytarabine, daunorubicin, or any chemotherapeutic. In some aspects, a therapeutic agent can be a siRNA, antisense oligonucleotide, or aptamers.

In some aspects, the nanoparticle comprises a cell-specific targeting moiety. In some aspects, the cell-specific targeting moiety can be a cancer cell-specific targeting moiety.

In some aspects, the targeting moiety can direct, or target, the nanoparticle to a specific cell. The cell-specific targeting moiety can be a chemical, compound, peptide or nucleic acid. Examples of targeting moieties include, but art not limited to, molecules that recognize receptors on specific cell types.

In some aspects, the nanoparticle can comprise a cell penetrating peptide. In some aspects, a cell penetrating peptide can facilitate the delivery of the nanoparticle to the cytoplasm of the cell.

In some aspects, the nanoparticle is labeled. As used herein, a label, or detection agent, is any molecule that can be associated with a nanoparticle, directly or indirectly, and which results in a measurable, detectable signal, either directly or indirectly. Many such labels for incorporation into nanoparticles or coupling to nanoparticles are known to those of skill in the art. Examples of detection agents can be, but are not limited to, radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluorophores are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

In some instances, a label can be, but is not limited to, an isotope marker, colorimetric biosensors, or fluorescent labels. For example, fluorescent markers can be, but are not limited to, green fluorescent protein (GFP) or rhodamine fluorescent protein (RFP). Other labels can include biotin, streptavidin, horseradish peroxidase, or luciferase.

In some aspects, the nanoparticle is a cationic-anionic polymer nanoparticle. The cationic-anionic make up of a nanoparticle allows for alternating electric fields to cause the high charge density cationic and anionic polymers to experience alignment and migration based on their charge, thus leading to disruption (or bursting) of the nanoparticle. In some aspects, the nanoparticle is a self-assembling cationic-anionic polymer nanoparticle. In some aspects, the cationic polymer can be chitosan or polyethylenimine (PEI), Poly (2-N,N-dimethylaminoethylmethacrylate-PDMAEMA), Poly (amidoamine) (PAMAM), polylysine-based polyplexes, Natural cationic polymers, cationic gelatin, cationic cellulose, cationic dextran. In some aspects, the anionic polymer can be bovine serum albumin (BSA), Polyacrylic acid cystamine conjugate, CMS-sodium carboxy methyl starch, CMG carboxy methyl guar gum, carboxymethyl cellulose, Sodium alginate.

In some aspects, the nanoparticle is 20-500 nm. In some aspects, the nanoparticle is 50-400 nm. In some aspects, the nanoparticle is 50-150 nm. In some aspects, the nanoparticle is 100-300 nm.

In some aspects, a device capable of administering an alternating electric field can be the Optune™ system.

### J. Kits

The materials described above as well as other materials can be packaged together in any suitable combination as a kit useful for performing, or aiding in the performance of, the disclosed methods. It is useful if the kit components in a given kit are designed and adapted for use together in the disclosed method. For example disclosed are kits comprising one or more of the disclosed nanoparticles. In some aspects, the kits can comprise the components to create the disclosed nanoparticles.

Disclosed are kits comprising a device capable of administering an alternating electric field and optionally a nanoparticle comprising a therapeutic agent. Disclosed are kits comprising a device capable of administering an alternating electric field and optionally a nanoparticle. In some aspects, the kits further comprise instructions for using the device and/or applying the alternating electrical field to a cell or a subject.

In some aspects, the kits disclosed herein can further comprise instructions for using a device capable of administering an alternating electric field in combination with a nanoparticle comprising a therapeutic agent.

In some aspects, the kits disclosed herein can comprise instructions for where to apply the alternating electrical field. In some aspects, the kits disclosed herein can comprise instructions for determining a region-of-interest (ROI) within a 3D model of a portion of a subject's body, determining, based on a center of the ROI, a plane that transverses the portion of the subject's body, wherein the plane comprises a plurality of pairs of positions along a contour of the plane, adjusting, based on an anatomical restriction, one or more positions of the plurality of pairs of positions to generate a modified plane, determining, for each pair of positions of the plurality of pairs positions on the modified plane, a simulated electric field distribution, determining, based on the simulated electric field distributions, a dose metric for each pair of positions of the plurality of pairs positions, determining one or more sets of pairs of positions of the plurality of pairs of positions that satisfy an angular restriction between pairs of transducer arrays, and determining, based on the dose metrics and the one or more sets of pairs of positions that satisfy the angular restriction, one or more candidate transducer array layout maps.

In some aspects, the kits disclosed herein comprise a device capable of administering an alternating electric field, wherein the kit further comprises electrodes for applying the alternating electric field (e.g. Optune system). In some aspects, the kits disclosed herein can further comprise instructions on where to apply the electrodes to increase the efficacy of alternating electric fields therapy. In some aspects, the kits disclosed herein further comprise instructions for conducting and analyzing measurements to determine where to apply the electrodes or where to apply the alternating electrical field.

### K. Embodiments

Embodiment 1. A method of delivering a therapeutic to a target site of a subject comprising: (A) administering a nanoparticle to a target site of a subject, wherein the nanoparticle comprises a therapeutic agent; and (B) applying an alternating electric field, at a frequency for a period of time, to the target site of the subject, wherein the alternating electric field releases the therapeutic from nanoparticle at the target site of the subject.

Embodiment 2. The method of any preceding embodiment, wherein the nanoparticle is loaded with the therapeutic agent.

Embodiment 3. The method of any preceding embodiment, wherein the therapeutic agent is encapsulated in the nanoparticle.

Embodiment 4. The method of any preceding embodiment, wherein the therapeutic agent is conjugated to the nanoparticle.

Embodiment 5. The method of any preceding embodiment, wherein the therapeutic agent is a small molecule, nucleic acid, carbohydrate, lipid, peptide, antibody, or antibody fragment.

Embodiment 6. The method of any preceding embodiment, wherein the nanoparticle is labeled.

Embodiment 7. The method of any preceding embodiment, wherein the nanoparticle is a cationic-anionic polymer nanoparticle.

Embodiment 8. The method of any preceding embodiment, wherein the nanoparticle is a self-assembling cationic-anionic polymer nanoparticle.

Embodiment 9. The method of any preceding embodiment, wherein the cationic polymer is chitosan or polyethylenimine (PEI).

Embodiment 10. The method of any preceding embodiment, wherein the anionic polymer is bovine serum albumin (BSA).

Embodiment 11. The method of any preceding embodiment, wherein the therapeutic is an anti-cancer therapeutic, an anti-viral therapeutic, an anti-bacterial therapeutic, an anti-fungal therapeutic, a pro-inflammatory therapeutic, or an anti-inflammatory therapeutic.

Embodiment 12. The method of embodiment 11, wherein the anti-cancer therapeutic is Gemcitabine (GEM).

Embodiment 13. The method of any preceding embodiment, wherein the nanoparticle comprises a cell-specific targeting moiety.

Embodiment 14. The method of embodiment 13, wherein the cell-specific targeting moiety is a cancer cell-specific targeting moiety.

Embodiment 15. The method of any preceding embodiment, wherein the nanoparticle enters the target site.

Embodiment 16. The method of any preceding embodiment, wherein the subject has cancer, an infection, an inflammatory disorder, or is immune suppressed.

Embodiment 17. The method of any preceding embodiment, wherein the target site comprises a cell.

Embodiment 18. The method of embodiment 17, wherein the cell is a cancer cell, a pathogen-infected cell, a mutant cell, or an immune cell.

Embodiment 19. The method of embodiment 18, wherein the cancer cell is a pancreatic cancer cell, glioblastoma cell, colon cancer cell, skin cancer cell, or lung metastatic carcinoma cell.

Embodiment 20. The method of embodiment 18, wherein the pathogen-infected cell is a virus infected, bacteria infected, or fungus infected cell.

Embodiment 21. The method of embodiments 18-20, wherein the drug-loaded nanoparticle enters the cancer cell or pathogen-infected cell.

Embodiment 22. The method of any preceding embodiment, further comprising applying a second alternating electric field at a second frequency to the target site prior to, during or after step a).

Embodiment 23. The method of embodiment 22, wherein the second alternating electric field increases permeability of a cell membrane of a cell at the target site of the subject.

Embodiment 24. The method of any preceding embodiment, wherein the nanoparticle is 20-500 nm.

Embodiment 25. The method of any preceding embodiment, wherein the frequency of the alternating electric field is between 100 and 500 kHz.

Embodiment 26. The method of any preceding embodiment, wherein the frequency of the alternating electric field is between 180 and 220 kHz.

Embodiment 27. The method of any preceding embodiment, wherein the nanoparticle is administered prior to, during or after exposing the target site to the alternating electric field.

Embodiment 28. A method of increasing target site specific release of a therapeutic agent in a subject comprising: (A) administering a nanoparticle to a target site of a subject, wherein the nanoparticle comprises a therapeutic agent; and (B) applying an alternating electric field, at a frequency for a period of time, to the target site of the subject, wherein the alternating electric field releases the therapeutic agent from the nanoparticle at the target site of the subject, thereby increasing the target site specific release of the therapeutic agent.

Embodiment 29. A method of treating a subject in need thereof comprising (A) administering a nanoparticle to a target site of a subject in need thereof, wherein the nanoparticle comprises a therapeutic agent; and (B) applying an alternating electric field, at a frequency for a period of time, to the target site of the subject in need thereof, wherein the alternating electric field releases the therapeutic agent from the nanoparticle at the target site of the subject in need thereof

Embodiment 30. The method of embodiment 29, wherein the subject in need thereof has cancer, an infection, or an inflammatory disorder.

Embodiment 31. The method of embodiment 30, wherein the infection is a viral, bacterial or fungal infection.

Embodiment 32. The method of embodiment 30, wherein the cancer is brain cancer, pancreatic cancer, lung cancer, ovarian cancer, colon cancer, skin cancer, or breast cancer.

Embodiment 33. A method of killing a cell comprising (A) administering a nanoparticle to a target site, wherein the nanoparticle comprises a therapeutic agent; and (B) applying an alternating electric field for a period of time, to the target site, wherein the alternating electric field releases the therapeutic agent from the nanoparticle at the target site, wherein the target site comprises a cell, wherein the therapeutic kills the cell.

Embodiment 34. The method of embodiment 33, wherein administering a nanoparticle to a target site comprises administering a nanoparticle to a target site of a subject.

Embodiment 35. The method of embodiments 33-34, wherein the cell is a cancer cell or a pathogen-infected cell.

Embodiment 36. The method of embodiments 28-35, wherein the nanoparticle is loaded with the therapeutic agent.

Embodiment 37. The method of embodiments 28-35, wherein the therapeutic agent is encapsulated in the nanoparticle.

Embodiment 38. The method of embodiments 28-35, wherein the therapeutic agent is conjugated to the nanoparticle.

Embodiment 39. The method of embodiments 28-38, wherein the therapeutic agent is a small molecule, nucleic acid, carbohydrate, lipid, peptide, antibody, or antibody fragment.

Embodiment 40. The method of embodiments 28-39, wherein the nanoparticle is labeled.

Embodiment 41. The method of embodiments 28-40, wherein the nanoparticle is a cationic-anionic polymer nanoparticle.

Embodiment 42. The method of embodiments 28-41, wherein the nanoparticle is a self-assembling cationic-anionic polymer nanoparticle.

Embodiment 43. The method of embodiments 41-42, wherein the cationic polymer is chitosan or polyethylenimine (PEI).

Embodiment 44. The method of embodiments 41-43, wherein the anionic polymer is bovine serum albumin (BSA).

Embodiment 45. The method of embodiments 28-45, wherein the therapeutic is an anti-cancer therapeutic, an anti-viral therapeutic, an anti-bacterial therapeutic, an anti-fungal therapeutic, a pro-inflammatory therapeutic, or an anti-inflammatory therapeutic.

Embodiment 46. The method of embodiment 45, wherein the anti-cancer therapeutic is Gemcitabine (GEM).

Embodiment 47. The method of embodiments 28-46, wherein the nanoparticle comprises a cell-specific targeting moiety.

Embodiment 48. The method of embodiments 47, wherein the cell-specific targeting moiety is a cancer cell-specific targeting moiety.

Embodiment 49. The method of embodiments 28-48, wherein the nanoparticle enters the target site.

Embodiment 50. The method of embodiments 28-49, wherein the subject has cancer, an infection, an inflammatory disorder, or is immune suppressed.

Embodiment 51. The method of embodiments 28-50, wherein the target site comprises a cell.

Embodiment 52. The method of embodiment 51, wherein the cell is a cancer cell, a pathogen-infected cell, or an immune cell.

Embodiment 53. The method of embodiment 52, wherein the cancer cell is a pancreatic cancer cell, glioblastoma cell, colon cancer cell, skin cancer cell, or lung metastatic carcinoma cell.

Embodiment 54. The method of embodiment 52, wherein the pathogen-infected cell is a virus infected, bacteria infected, or fungus infected cell.

Embodiment 55. The method of embodiments 52-54, wherein the nanoparticle enters the cancer cell or pathogen-infected cell.

Embodiment 56. The method of embodiments 28-55, further comprising applying a second alternating electric field at a second frequency to the target site prior to, during or after step a).

Embodiment 57. The method of embodiment 56, wherein the second alternating electric field increases permeability of a cell membrane of a cell at the target site of the subject.

Embodiment 58. The method of embodiments 28-57, wherein the nanoparticle is 20-500 nm.

Embodiment 59. The method of embodiments 28-58, wherein the frequency of the alternating electric field is between 100 and 500 kHz.

Embodiment 60. The method of embodiments 28-59, wherein the frequency of the alternating electric field is between 180 and 220 kHz.

Embodiment 61. The method of embodiments 28-60, wherein the nanoparticle is administered prior to, during or after exposing the target site to the alternating electric field.

Embodiment 62. A drug delivery system comprising: (A) a nanoparticle comprising a therapeutic agent; and (B) a device capable of administering an alternating electric field.

Embodiment 63. A kit comprising: (A) a nanoparticle comprising a therapeutic agent; and (B) a device capable of administering an alternating electric field.

Embodiment 64. The method of embodiments 62-63, further comprising instructions for using the device.

### EXAMPLES

### A. Example 1: Tumor Treating Fields Triggered Targeting of Nanoparticles in Cancer

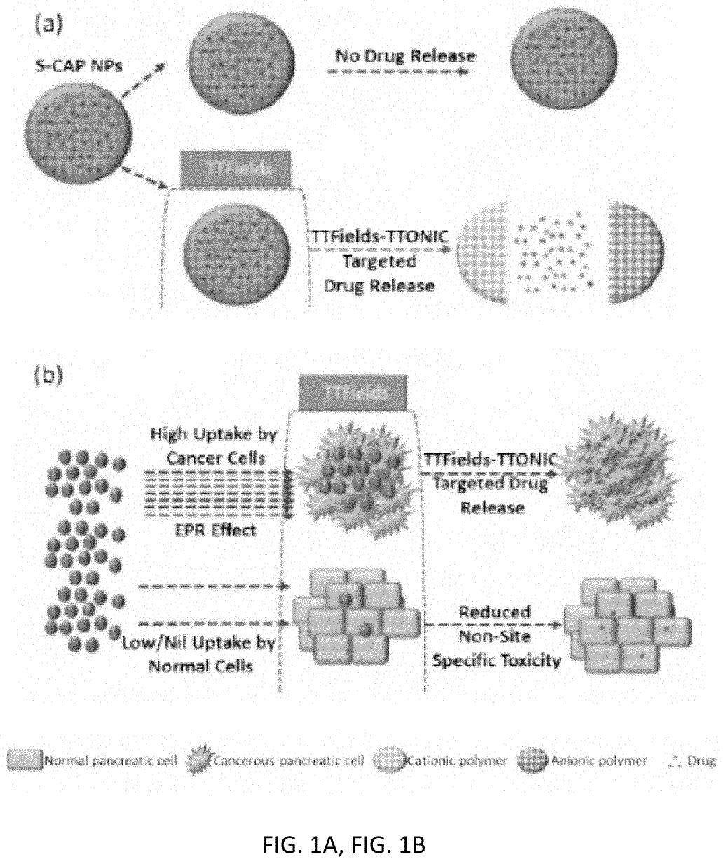

The use of novel nanoformulations of anticancer/chemopreventive agents and their combinations towards PC treatment and prevention are actively being studied. The importance of delivering these agents using a novel nanotechnology-based lipid, polymeric and lipo-polymeric system wherein high efficacy was achieved at extremely low doses has been demonstrated (8, 13-15). ‘Tumor Treating Fields Triggered Targeting of Nanoparticles in Cancer (TTFields-TTONIC)’ is the approach disclosed herein. TTFields stimuli sensitive anticancer drug loaded nanoparticles (NPs) are developed which will enable tumor site-specific drug release in the presence of locally applied TTFields. A schematic of a proposed strategy is depicted in FIGS. 1A and B.

In principle, TTFields employ low intensity, intermediate frequency, alternating electric fields (100-300 KHz) to disrupt the mitotic tumor cancer cell division. Mechanistically, under the applied electric field the random motion of high charge density macromolecules (tubulin, septin etc.) is restricted by dipole alignment (alignment of polar macromolecules parallel to applied electric field) and dielectrophoresis (migration toward a region of high-field density). This restriction of random motion interferes with the cell division (mitosis, cytokinesis) leading to the tumor cells autophagy (5, 16, 17).

The similar principle can be applied to high charged density polymers. Specifically, self-assembling cationic-anionic polymer nanoparticles (S-CAP NPs) can be used. The cationic polymers investigated are Chitosan and Polyethylenimine (PEI), whereas the anionic polymer is Bovine Serum Albumin (BSA) and Gemcitabine (GEM) as a model anticancer drug for PC. Based on the electrostatic interaction between cationic and anionic polymers, stable S-CAP NPs encapsulating GEM are formulated. In the presence of locally applied TTFields, high charge density cationic and anionic polymers can experience alignment and migration based on their charge and the electric field leading to destabilization of S-CAP NPs assembly thus leading to disruption of NPs and thereby releasing the drug at the tumor site (FIG. 1A). The particle size of S-CAP NPs is optimized between 200-400 nm for their preferential uptake in cancer cells by means of tumor specific leaky vasculature ensuring enhanced permeation and retention (EPR) effect (18-23). Considering high uptake by tumor cells combined with TTFields triggered targeted drug release from S-CAP NPs, tumor specific drug release can be ensured. Further non-site specific drug release can be reduced and hence subsequent adverse effects can be reduced.

### 1. Results

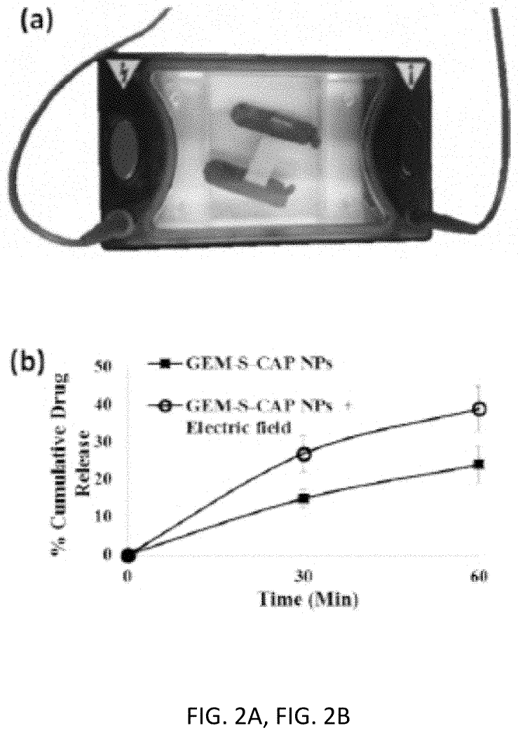

GEM encapsulated chitosan-BSA S-CAP NPs were developed using solvent assisted ionic gelation method (particle size: 478.64 nm±58.29 nm, PDI: 0.383). The developed formulation was subjected to in vitro dissolution studies using the dialysis bag method and an electrophoresis device in the presence and absence of an electric field. The % drug release was calculated at the end of 30 min and 60 min. The device assembly and the results are depicted in FIG. 2A and FIG. 2B respectively. The studies demonstrated almost 1.5-fold increase in the drug release at the end of 60 min. In summary, the specific advantages of the proposed strategy are: 1. A combination of TTFields with TTFields-TTONIC; 2. Reduction in non-site specific release of anticancer drugs thereby reducing adverse effects; 3. reducing the existing dose of anticancer drugs; 4. Use in cancer chemoprevention and other types of cancer.

### 2. Research Design and Methods

### i. Development and Optimization of GEM Encapsulated S-CAP NPs

### a. Rationale and Overview of the Study Design

S-CAP NPs offer key advantages like high uptake by cancer cells, site specific stimuli sensitive controlled drug release, drug encapsulation, good tolerability, easy scalability and high bioavailability. Described herein is the use of S-CAP NPs to enable TTFields triggered drug release at the cancer site. However, formulation optimization (specifically the NP size between 200-700 nm, polymer composition) is necessary towards the success of TTFields-TTONIC modality in PC. Studies can be performed to determine the development and optimization of GEM encapsulated S-CAP NPs with respect to polymer composition, size, polydispersity index, zeta potential, encapsulation efficiency and drug release and to conduct detailed physicochemical characterization of developed S-CAP NPs as well as to conduct stability studies as per ICH guidelines to ensure formulation stability.

### b. Research Design and Methods

(A) Development and Optimization of GEM Encapsulated S-CAP NPs

Multiple batches of Chitosan-BSA and PEI-BSA S-CAP NPs can be prepaid using solvent assisted ionic gelation/solvent emulsion evaporation method using Quality by Design approach (QbD). Briefly, the GEM and polymer mixture can be solubilized in the suitable solvent followed by addition of stabilizer containing aqueous phase. Once the homogenous mixture is obtained the solvent phase can be evaporated resulting in the formation of NPs. Herein the ratio of cationic to anionic polymer, drug loading, solvent concentration can be optimized as an independent variables and size, zeta potential, encapsulation efficiency, drug release can be studied as dependent variables. Based on the developed mathematical model a control strategy can be designed and suitable S-CAP NPs can be selected.

(B) Physicochemical Characterization

Particle size, polydispersity index (PDI) and zeta potential analysis: A Zetasizer (Nano-ZS; Malvern Instruments) and software, Dispersion Technology Software can be used.

In vitro drug release studies: The studies can be performed using dialysis bag method. Samples can be withdrawn periodically and analyzed using a validated HPLC method

Encapsulation efficiency: S-CAP NPs can be suspended in an appropriate solvent and encapsulation efficiency determined by analyzing the supernatant for un-encapsulated drug.

Electron microscopy imaging: SEM and TEM imaging allows the understanding of morphological characteristics of the formulation.

FTIR and XRD analysis: This allows identification and understanding of interactions between drug and excipients.

(C) Stability Studies:

These studies can be conducted as per the ICH guidelines wherein the sample subjected to stability storage can be assessed at predetermined time points for size, zeta potential, encapsulation efficiency and drug release. This allows valuable information regarding the stability of formulation under proposed storage conditions (room temperature/refrigeration etc.)

### ii. Study the Uptake, Internalization and Intracellular Localization of S-CAP NPs

### a. Rationale and Overview of the Study Design

S-CAP NPs can exhibit enhanced and selective intracellular uptake by the PC cells owing to the tumor specific leaky vasculature. This enhanced intracellular uptake can increase the concentration of S-CAP NPs in the PC tumors. Once concentrated in the PC tumors, the TTFields induction ensures the drug release from the S-CAP NPs. Hence, it is important to study the cellular internalization and localization of proposed S-CAP NPs in PC cells. Comparing the PC cells uptake potential of two different S-CAP NPs can also be done. In vitro subcellular localization S-CAP NPs in PC cell lines using confocal microscopy and flow cytometry analysis as well as comparison of in vitro uptake of chitosan-BSA and PEI-BSA S-CAP NPs to select the optimum S-CAP NPs formulation can be performed.

### b. Research Design and Methods

(A) Cell Lines:

Studies can be executed in 2 phases. In Phase 1, PC cancer cell lines Mia PaCa-2 and Panc-1 can be studied. In phase 2, K-ras wild-type normal pancreatic epithelial cells (HPNE-hTERT) and K-ras transformed cells (hTERT-HPNE E6/E7/K-RasG12D/st) can be studied as more advanced cell line model. The cell lines can be procured from ATCC and can be authenticated using recommended tests viz. microscopic morphology test, growth curve analysis, mycoplasma testing and overruling presence of any cross-contamination. The cell lines can be maintained as per specified protocols.

(B) Intracellular Uptake of S-CAP NPs by PC Cell Lines:

The studies can be conducted using fluorescence method (15). To formulate fluorescent dye loaded S-CAP NPs, octadecylamine-fluoroscein isothiocynate (ODA—FITC) dye can be encapsulated in the S-CAP NPSs followed by centrifugation. The fluorescent S-CAP NPs can be incubated with PC cell lines in wells chamber slides for varied time period (5, 30, 60, 120 min and 24 h), cells can then be washed with PBS. The cells can be observed under a fluorescence microscope (Nikon, Japan) at excitation wavelength of 490 nm. The cellular uptake percentage of fluorescent S-CAP NPs can be calculated from equation:

Cellular uptake of fluorescent S-Cap NPs (%)=I/I0×100%‘ where I is the fluorescence intensity at different time points and I0 is the initial intensity of the fluorescent S-CAP NPs.

(C) In Vitro Subcellular Localization S-CAP NPs in PC Cell Lines:

Both (chitosan-BSA and PEI-BSA) S-Cap NPs can be labelled with a fluorescent dye for real time tracking of cellular internalization. Fluorescein-labeling of S-CAP NPs can be executed based on the literature reported method and our prior experience (15). The labelled S-CAP NPs can be incubated with PC cell lines in wells chamber slides for varied time period (5, 30, 60, 120 min and 24 h), cells can then be washed with PBS, followed labelling with CellLight Blue DND-22 (blue color) to tag endosomes and lysosome and fixed with 4% formaldehyde for 15 min. Images can be acquired using a Nikon microscope.

### iii. Conduct in Vitro Tumor Cell Line, Colony Formation Inhibition Studies of GEM Encapsulated S-CAP NPs in Presence of TTFields for TTFields-TTONIC Modality

### a. Rationale and Overview of the Study Design

Frequency of TTFields and S-CAP NPs properties (cationic and anionic polymer composition, zeta potential, drug encapsulation) can impact the stimuli sensitive drug release from the S-CAP NPs. It is reported that 100-300 KHz frequency is used during TTField treatment and 150 KHz frequency is considered optimum for PC treatment (2, 5). Hence, the effect of TTField frequency on both PEI-BSA and Chitosan-BSA S-CAP NPs to select optimum frequency and the superior S-CAP NPs for maximum efficacy can be determined.

Research design and methods:

(A) Cell Lines:

Same cell lines as used for ii(b)(A) above.

(B) Cell Inhibition MTS Assay:

Tumor cell growth inhibition will be assessed by MTS assay. Briefly, cells will be treated individually with GEM loaded chitosan-BSA and PEI-BSA S-CAP NPs in presence of varied frequency TTFields using Inovitro™ TTFields Lab Bench System for a period of 72 h. The relative quantification of viable cells will be determined compared to the control using MTS assay and cell inhibition will be measured. Also, IC50 measurements will be calculated (13, 38). Based on the results, optimum combination of TTFields frequency and superior S-CAP NPs can be selected.

(C) Colony Formation Assay:

The study will be executed by soft agar colony formation assay. Briefly, cells will be treated individually with GEM loaded chitosan-BSA and PEI-BSA S-CAP NPs in presence of varied frequency TTFields using Inovitro™ TTFields Lab Bench System for period of 72. Cells will then be collected and plated in 6-well tissue culture plates contained agarose medium. At the end of 2 weeks, the colonies will be stained with 0.5% crystal violet and number of colonies will be counted (38, 39). The inhibition can be determined by comparing with control. Based on the results, optimum combination of TTFields frequency and superior S-CAP NPs can be selected.

### iv. Study the In Vivo Toxicity of S-CAP NPs in a Preclinical Model

### a. Rationale and Overview of the Study Design

Safety, efficacy are key features of any pharmaceutical drug delivery system to be successful, and the polymeric nanoparticles proposed in our project have solid literature support. Chitosan and BSA are biocompatible and biodegradable polymers and have been used clinically for many years (24-37). However, the unique feature of the proposed S-CAP NPs is the concurrent use of cationic and anion polymers. The most optimum S-CAP NPs can be selected based on the results above. Acute and repeated dose 28-day toxicity of the selected S-CAP NPs in Sprague-Dawley (SD) rats can be performed.

### b. Research Design and Methods

(A) Acute toxicity and repeated dose 28-day toxicity study of S-CAP NPs in vivo

Acute toxicity studies can be performed as per the OECD guidelines wherein 3 female SD rats can be used for each step along with control. Based on recommendations, the starting dose can be selected as one of four fixed levels, 5, 50, 300 and 2000 mg/kg. Animals will be observed during first 30 min followed by periodic observation up to 24 h and then daily up to 14 days. Body weight, any observation regarding abnormal behavior or death can be reported. Necropsy can be performed at the end of the study to examine internal organs and LD50 concentrations will be determined (40). Repeated dose 28-day toxicity study in rodents can be performed as per the OECD guidelines to assess toxic effect on various organs and to establish no-observed adverse effect level (NOAEL) (41). For this, 10 (five female and five male) SD rats can be used for each step. Based on the results of acute toxicity study, if highest dose is safe and no effect is expected at a dose of 1000 mg kg body weight/d, a limit test can be executed. If not, minimum 3 test group can be used along with control. Body weight, any observation regarding abnormal behavior or death will be reported. Necropsy can be performed at the end of the study to examine internal organs and NOAEL will be determined. Maximum 35 female SD rats (3*5=15 female SD rats for acute toxicity study and 5*4=20 female SD rats for repeated dose 28-day toxicity study) and maximum 20 male SD rats (5*4=20 male SD rats for repeated dose 28-day toxicity study) will be used. Animal number may decrease based on observations.

### c. Statistical Analysis

All the studies can be executed in triplicate. Results can be statistically analyzed by the one way analysis of variance (ANOVA) followed by appropriate test using GraphPad Prism 6 (GraphPad Software, La Jolla, Calif.) software (significance at *P<0.05, **P<0.01, and ***P<0.001).

### 3. Conclusions

Success of TTFields-TTONIC modality of PC management can open new potential avenues in management of PC and other cancer types in future. It will not only ensure the use of TTFields as an antimitotic tumor inhibitor but also as a stimuli device for targeted drug delivery of anticancer drugs. This concurrent use of TTFields and our proposed TTFields-TTONIC can enhance the overall treatment efficacy with significant reduction in side effects and hence can provide a potentially viable solution in cancer treatment.

### B. Example 2: Drug Loaded Nanoparticle Targeting of Pancreatic Cancer Using Tumor Treating Fields (TTFields)

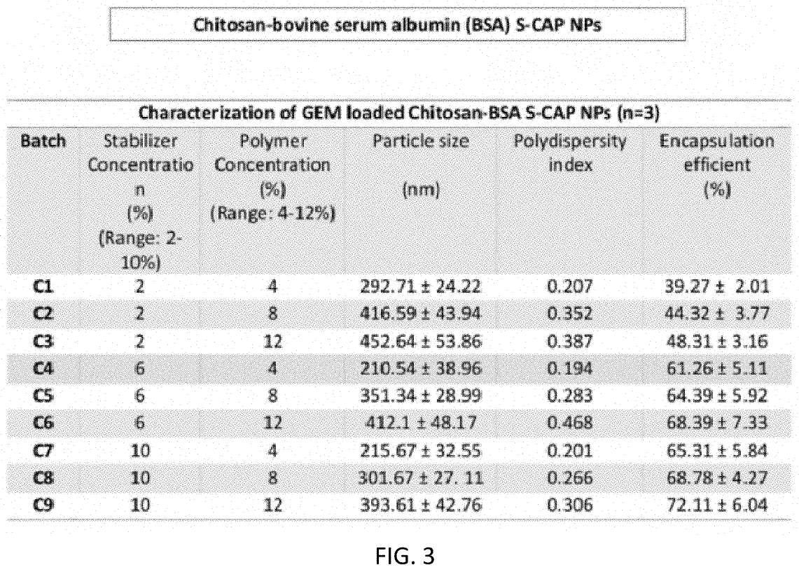

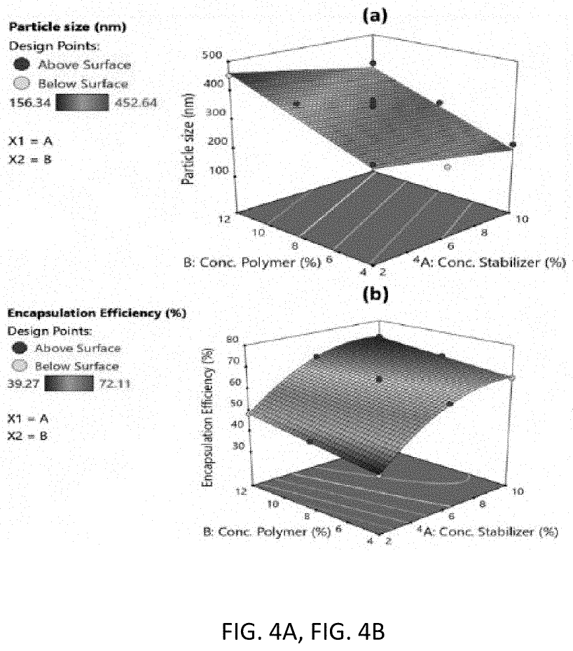

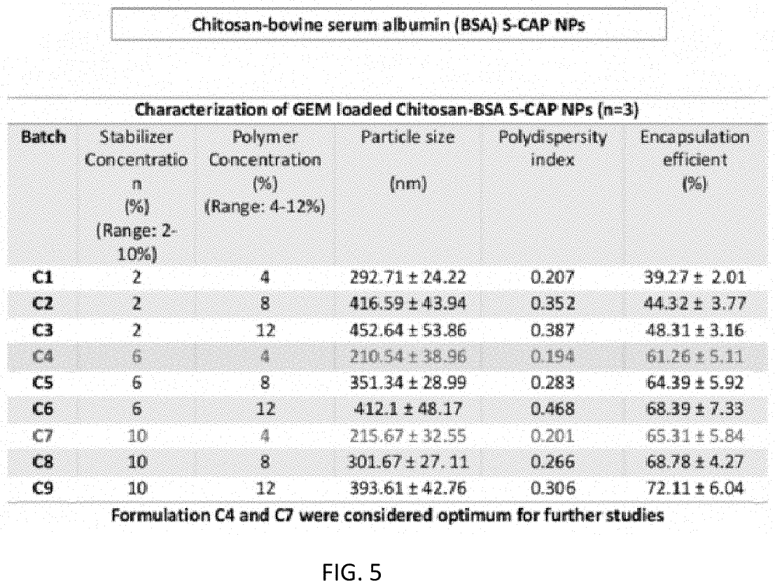

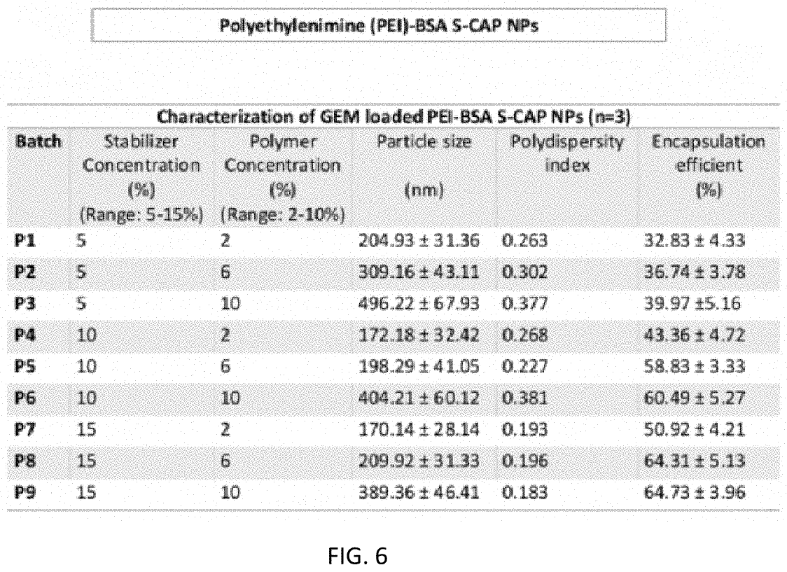

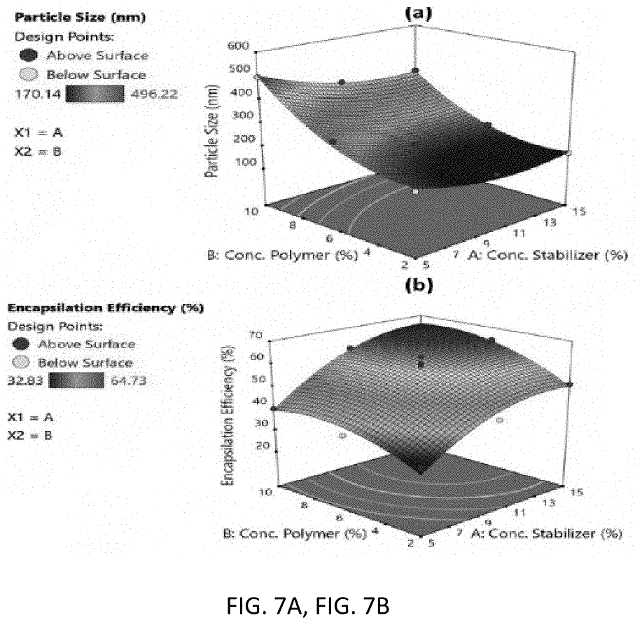

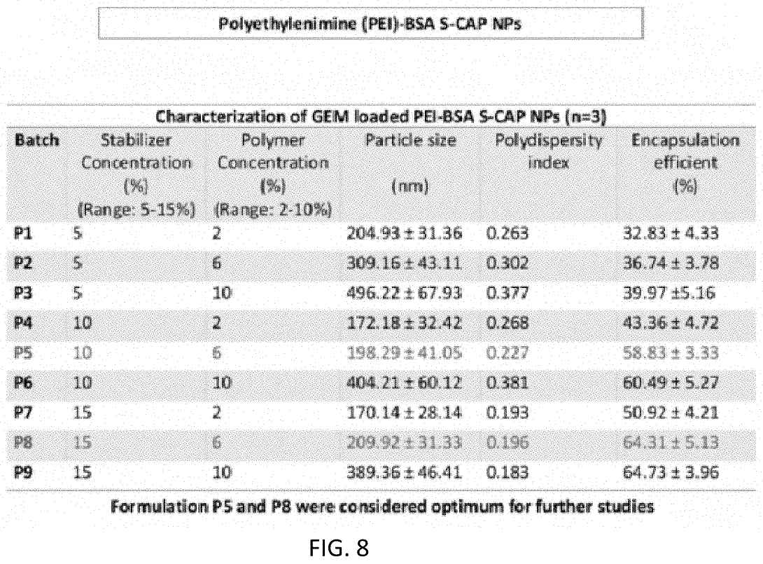

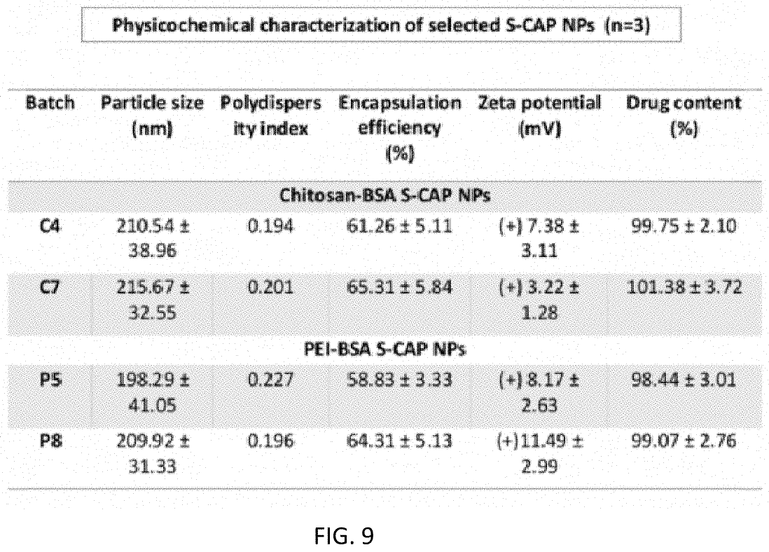

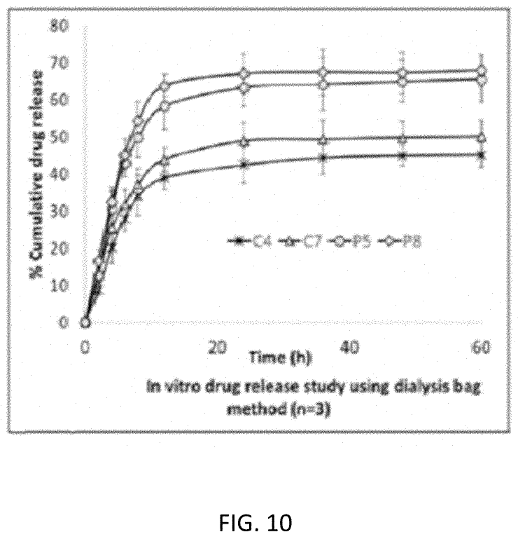

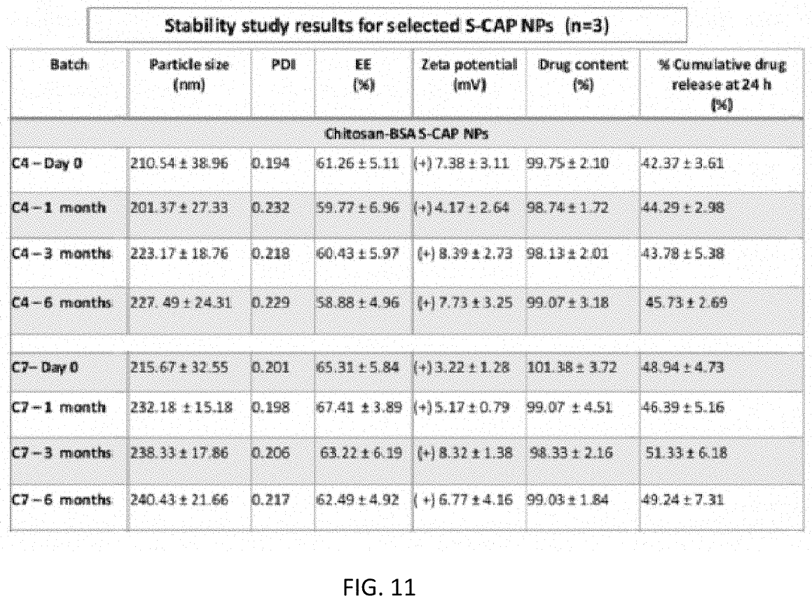

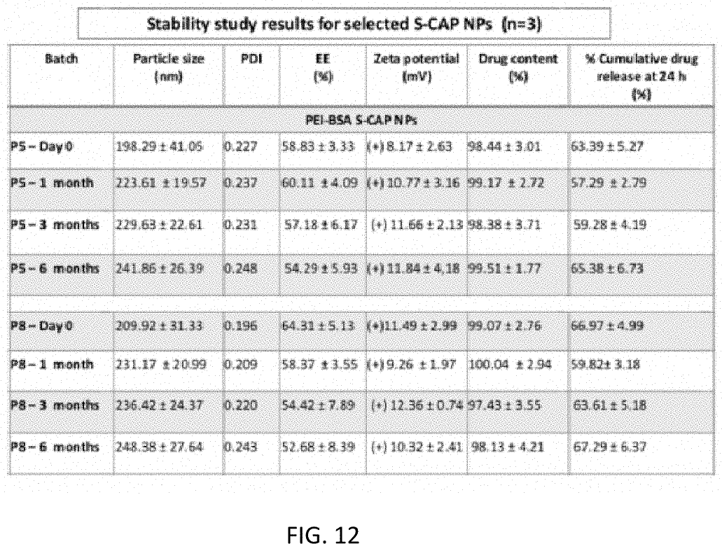

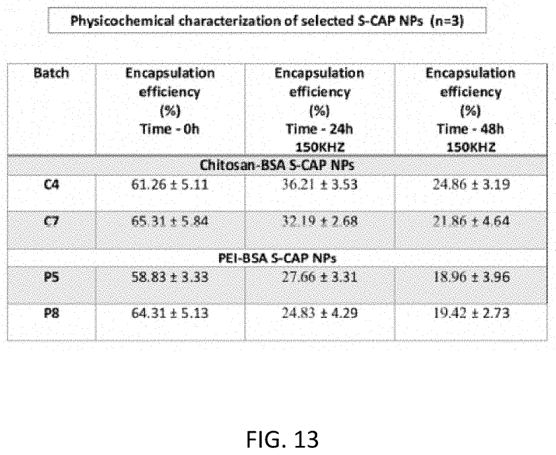

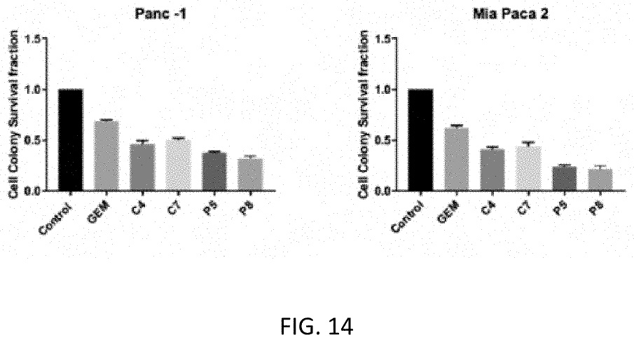

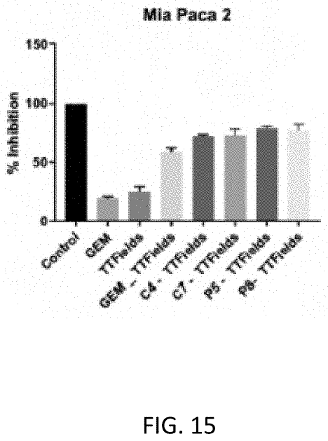

TTFields have been clinically proven as safe, effective, and non-invasive approach for cancer treatment. Specifically, TTFields in conjunction with Gemcitabine/nab-Paclitaxel have shown promising results in Phase II pancreatic cancer (PC) PANOVA study. Concurrent use of anticancer drugs can continue to elicit non-site-specific adverse effects resulting in overall low patient compliance. To overcome this drawback, a strategy called ‘Tumor Treating Fields Triggered Targeting of Nanoparticles in Cancer (TTFields-TTONIC) was developed. For this, self-assembling cationic-anionic polymer nanoparticles (S-CAP NPs) encapsulating Gemcitabine as a model anticancer drug were developed. The combination of NPs and TTFields can be used wherein the developed NPs can be preferentially taken up by the tumor owing to leaky vasculature. Further, only under the applied TTFields, the NPs can be destabilized due to high charge density of cationic and anionic polymers leading to targeted release of encapsulated drug at the tumor site (reduction in non-site-specific side effects). For this, multiple batches of two types of S-CAP NPs [chitosan-bovine serum albumin (Chitosan-BSA) and polyethylenimine-bovine serum albumin (PEI-BSA)] were developed. The formulations were optimized using mathematical modelling and Design Expert® software to achieve low particle size and optimum encapsulation efficiency. Based on the results, 2 formulations from each type i.e., chitosan-BSA S-CAP NPs [Batch C4-particle size: 210.54±38.96 nm, PDI: 0.194, encapsulation efficiency: 61.26±5.11%, zeta potential: (+) 7.38 ±3.11; Batch C7-particle size: 215.67±32.55 nm, PDI: 0.201, encapsulation efficiency: 65.31±5.84%, zeta potential: (+) 3.22±1.28] and PEI-BSA S-CAP NPs [Batch P5—particle size: 198.2935 41.05 nm, PDI: 0.227, encapsulation efficiency: 58.83±3.33%, zeta potential: (+) 8.17±2.63; Batch P8—particle size: 209.92±31.33 nm, PDI: 0.196, encapsulation efficiency: 64.31 ±5.13%, zeta potential: (+) 11.49±2.99] were shortlisted that exhibited particle size—200 nm and encapsulation efficiency in range of 55-65%. (See FIGS. 3-9 and FIG. 13). All the formulations exhibited sustained drug release profile over a period of 60 h wherein chitosan-BSA S-CAP NPs showed slower drug release compared to PEI-BSA S-CAP NPs (P<0.05) (FIG. 10). All formulations were subjected to stability studies as per the ICH guidelines wherein the formulations were observed to be stable. (FIG. 11 and FIG. 12) Further, the conventional MTS inhibitory assay and colony formation assay against the PC cell lines (Panc-1 and Mia Paca-2) showed significant efficacy with the PEI-BSA S-CAP NPs (P<0.01) compared to chitosan-BSA S-CAP NPs. (FIG. 14 and FIG. 15). In the next phase, efficacy of S-CAP NPs against the PC cell lines in presence of TTFields can be analyzed followed by in vivo safety studies. The success of this strategy can enhance the safety of combination treatment involving TTFields-anticancer drugs and also establish a newer application of TTFields as a targeting modality.

### C. Example 3

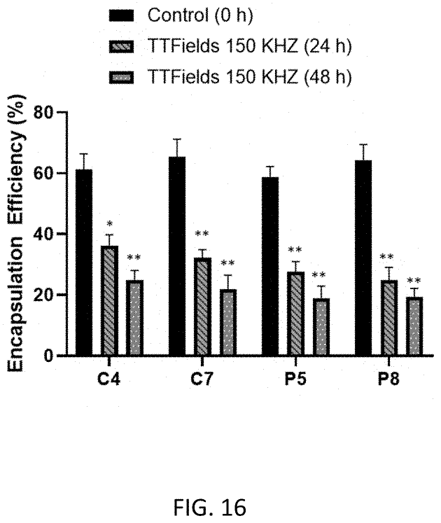

Encapsulation efficiency is examined in FIG. 16. After treatment with TTFields for 24 or 48 hrs, the encapsulation efficiency decreased significantly compared to nanoparticles that did not receive TTFields. Thus, the TTFields caused a release of the drug that was in the nanoparticle.

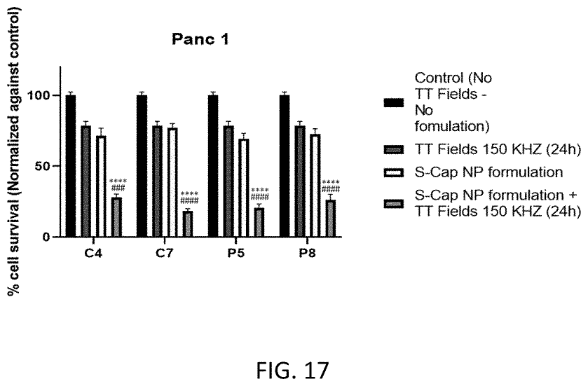

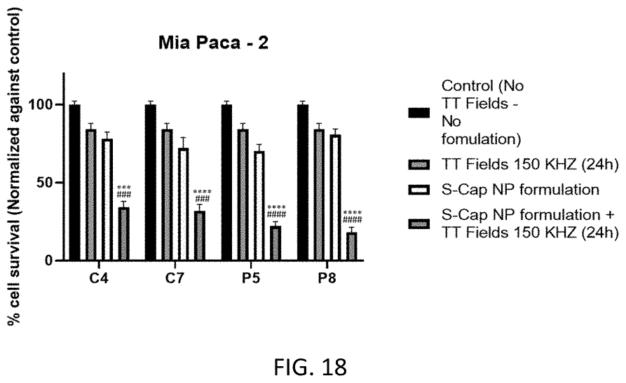

FIG. 17 and FIG. 18 show that the combination of TTFields and S-Cap NPs caused a significant decrease in cell survival. This indicates that the TTFields helped burst the nanoparticles allowing the drug to be delivered to the cell and kill the cell. Synergism in inhibition was observed when pancreatic cancer cell lines were treated with S-CAP NPs in presence of TTFields. The studies confirmed targeted drug delivery triggered by TTFields. In the Mia Paca-2 cell line (FIG. 18), higher efficacy was observed with PEI-BSA S-CAP NPs compared to chitosan-BSA S-CAP NPs.

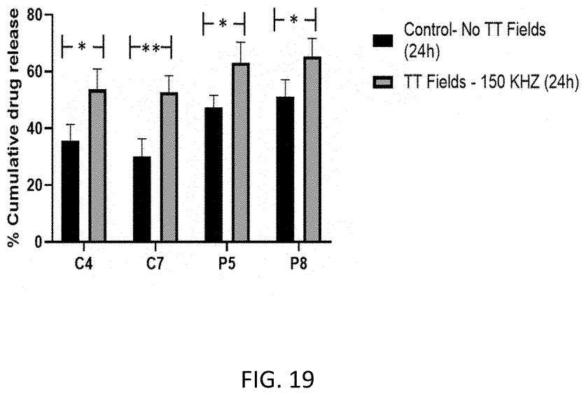

The effect of TTFields (150 KHz) on drug release profile of S-Cap-NPs at end of 24 hours was studied using TTFields and high wall dishes (FIG. 19). The studies revealed that TTFields trigger faster drug release from S-Cap-NPs by destabilizing the NPs. All the formulations showed a significant increase in the drug release in the presence of TTFields compared to respective control (Formulation not subjected to TTFields). Among all S-Cap NPs, formulation C7 showed highest increase in rate of drug release (p<0.01) in presence of TTFields. The studies confirmed TTFields trigger drug release from S-Cap-NPs.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. USFDA. NovoTTF-100A System—Premarket approval P100034. 2011 [2/6/2019]; Available from: www.accessdata.fda.gov/scripts/cdrh/cfdocs/cfpma/pma.cfm?id=P100034.
2. Benavides M, Guillen C, Rivera F, Gallego J, Lopez-Martin J A, Küng M. PANOVA: A phase II study of TTFields (150 kHz) concomitant with standard chemotherapy for front-line therapy of advanced pancreatic adenocarcinoma—Updated efficacy results. Journal of Clinical Oncology. 2017;35(15_suppl):e15790-e.
3. Giladi M, Schneiderman R S, Porat Y, Munster M, Itzhaki A, Mordechovich D, et al. Mitotic disruption and reduced clonogenicity of pancreatic cancer cells in vitro and in vivo by tumor treating fields. Pancreatology. 2014 Jan-Feb;14(1):54-63.
4. Giladi M, Weinberg U, Schneiderman R S, Porat Y, Munster M, Voloshin T, et al. Alternating electric fields (tumor-treating fields therapy) can improve chemotherapy treatment efficacy in non-small cell lung cancer both in vitro and in vivo. Semin Oncol. 2014 Oct;41 Suppl 6:S35-41.
5. Mun E J, Babiker H M, Weinberg U, Kirson E D, Von Hoff D D. Tumor-Treating Fields: A Fourth Modality in Cancer Treatment. Clin Cancer Res. 2018 Jan. 15;24(2):266-75.
6. Vergote I, Moos Rv, Manso L, Sessa C. INNOVATE: A phase II study of TTFields (200 kHz) concomitant with weekly paclitaxel for recurrent ovarian cancer—Updated safety and efficacy results. Journal of Clinical Oncology. 2017;35(15_suppl):5580—.
7. Von Hoff D D, Ervin T, Arena FP, Chiorean E G, Infante J, Moore M, et al. Increased survival in pancreatic cancer with nab-paclitaxel plus gemcitabine. N Engl J Med. 2013 Oct. 31;369(18):1691-703.
8. Desai P, Ann D, Wang J, Prabhu S. Pancreatic Cancer: Recent Advances in Nanoformulation-Based Therapies. 2019 2018-10-09;36(1):59-91.
9. AmericanChemicalSociety. Key Statistics for Pancreatic Cancer. 2019 [2/6/2019]; Available from: www. cancer. org/cancer/pancreatic-cancer/about/key-statistics.html.
10. Fischer R, Breidert M, Keck T, Makowiec F, Lohrmann C, Harder J. Early recurrence of pancreatic cancer after resection and during adjuvant chemotherapy. Saudi J Gastroenterol. 2012 Mar-Apr;18(2):118-21.
11. TexasOncology. Recurrent Pancreatic Cancer. 2019 [cited 2019 2/6/2019]; Available from: https://www.texasoncology.com/types-of-cancer/pancreatic-cancer/recurrent-pancreatic-cancer.
12. Rivera F, Gallego J, Guillen C, Benavides M, Lopez-Martin J A, Betticher D C, et al. PANOVA: A pilot study of TTFields concomitant with gemcitabine for front-line therapy in patients with advanced pancreatic adenocarcinoma. Journal of Clinical Oncology. 2016;34(4_suppl):269-.
13. Desai P, Thakkar A, Ann D, Wang J, Prabhu S. Loratadine self-microemulsifying drug delivery systems (SMEDDS) in combination with sulforaphane for the synergistic chemoprevention of pancreatic cancer. Drug Deliv Transl Res. 2019 Jan 31.
14. Desai P, Thakkar A, Wang J, Prabhu S. Abstract 2238: Self-microemulsifying drug delivery systems (SMEDDS) containing novel compounds for the chemoprevention of pancreatic cancer. Cancer Research. 2018;78(13 Supplement):2238—.
15. Thakkar A, Desai P, Chenreddy S, Modi J, Thio A, Khamas W, et al. Novel nano-drug combination therapeutic regimen demonstrates significant efficacy in the transgenic mouse model of pancreatic ductal adenocarcinoma. Am J Cancer Res. 2018;8(10):2005-19.
16. Giladi M, Schneiderman R S, Voloshin T, Porat Y, Munster M, Blat R, et al. Mitotic Spindle Disruption by Alternating Electric Fields Leads to Improper Chromosome Segregation and Mitotic Catastrophe in Cancer Cells. Sci Rep. 2015 Dec 11;5:18046.
17. Wenger C, Salvador R, Basser P J, Miranda P C. The electric field distribution in the brain during TTFields therapy and its dependence on tissue dielectric properties and anatomy: a computational study. Phys Med Biol. 2015 Sep 21;60(18):7339-57.
18. Azzi S, Hebda J K, Gavard J. Vascular permeability and drug delivery in cancers. Front Oncol. 2013;3:211.
19. Barua S, Mitragotri S. Challenges associated with Penetration of Nanoparticles across Cell and Tissue Barriers: A Review of Current Status and Future Prospects. Nano Today. 2014 Apr. 1;9(2):223-43.
20. Gradishar W J, Tjulandin S, Davidson N, Shaw H, Desai N, Bhar P, et al. Phase III trial of nanoparticle albumin-bound paclitaxel compared with polyethylated castor oil-based paclitaxel in women with breast cancer. J Clin Oncol. 2005 Nov 1;23(31):7794-803.
21. Hashizume H, Baluk P, Morikawa S, McLean J W, Thurston G, Roberge S, et al. Openings between defective endothelial cells explain tumor vessel leakiness. Am J Pathol. 2000 Apr;156(4):1363-80.
22. Hobbs S K, Monsky WL, Yuan F, Roberts W G, Griffith L, Torchilin V P, et al. Regulation of transport pathways in tumor vessels: role of tumor type and microenvironment. Proc Natl Acad Sci U S A. 1998 Apr 14;95(8):4607-12.
Nakamura Y, Mochida A, Choyke P L, Kobayashi H. Nanodrug Delivery: Is the Enhanced Permeability and Retention Effect Sufficient for Curing Cancer? Bioconjugate Chemistry. 2016 2016/10/19;27(10):2225-38.
24. Balint R, Cassidy N J, Cartmell S H. Conductive polymers: Towards a smart biomaterial for tissue engineering. Acta Biomaterialia. 2014 2014/06/01/;10(6):2341-53.
25. Guan Y-G, Lin H, Han Z, Wang J, Yu S-J, Zeng X-A, et al. Effects of pulsed electric field treatment on a bovine serum albumin—dextran model system, a means of promoting the Maillard reaction. Food Chemistry. 2010 2010/11/15/;123(2):275-80.
26. Kim S J, Ryon Shin S, Han Lee J, Hoon Lee S, I. Kim S. Electrical response characterization of chitosan/polyacrylonitrile hydrogel in NaCl solutions2003. 91-6 p.
27. Mohapatra A, McGraw G, Morshed B I, Jennings J A, Haggard W O, Bumgardner J D, et al., editors. Electric stimulus response of chitosan microbeads embedded with magnetic nanoparticles for controlled drug delivery. 2014 IEEE Healthcare Innovation Conference (HIC); 2014 8-10 Oct. 2014.
28. Hu Y L, Qi W, Han F, Shao J Z, Gao J Q. Toxicity evaluation of biodegradable chitosan nanoparticles using a zebrafish embryo model. Int J Nanomedicine. 2011;6:3351-9.
29. Mohammed M A, Syeda J T M, Wasan K M, Wasan E K. An Overview of Chitosan Nanoparticles and Its Application in Non-Parenteral Drug Delivery. Pharmaceutics. 2017 Nov. 20;9(4).
30. Pang Z, Gao H, Chen J, Shen S, Zhang B, Ren J, et al. Intracellular delivery mechanism and brain delivery kinetics of biodegradable cationic bovine serum albumin-conjugated polymersomes. Int J Nanomedicine. 2012;7:3421-32.
31. An F F, Zhang X H. Strategies for Preparing Albumin-based Nanoparticles for Multifunctional Bioimaging and Drug Delivery. Theranostics. 2017;7(15):3667-89.
32. Babaei M, Eshghi H, Abnous K, Rahimizadeh M, Ramezani M. Promising gene delivery system based on polyethylenimine-modified silica nanoparticles. Cancer Gene Therapy. 2017 01/27/online;24:156.
33. Comas-Rojas H, Enriquez-Victorero C, Roser S J, Edler K J, Pérez-Gramatges A. Self-assembly and phase behaviour of PEI : cationic surfactant aqueous mixtures forming mesostructured films at the air/solution interface. Soft Matter. 2013;9(15):4003-14.
34. Jiang D, Wang M, Wang T, Zhang B, Liu C, Zhang N. Multifunctionalized polyethyleneimine-based nanocarriers for gene and chemotherapeutic drug combination therapy through one-step assembly strategy. Int J Nanomedicine. 2017;12:8681-98.
35. Lee J E, Kim M G, Jang Y L, Lee M S, Kim NW, Yin Y, et al. Self-assembled PEGylated albumin nanoparticles (SPAN) as a platform for cancer chemotherapy and imaging. Drug Deliv. 2018 Nov;25(1):1570-8.
36. Lungwitz U, Breunig M, Blunk T, Gopferich A. Polyethylenimine-based non-viral gene delivery systems. European Journal of Pharmaceutics and Biopharmaceutics. 2005 2005/07/01/;60(2):247-66.
37. Wang Y, Xu S, Xiong W, Pei Y, Li B, Chen Y. Nanogels fabricated from bovine serum albumin and chitosan via self-assembly for delivery of anticancer drug. Colloids Surf B Biointerfaces. 2016 Oct 1;146:107-13.
38. Giladi M, Weinberg U, Schneiderman R S, Porat Y, Munster M, Voloshin T, et al. Alternating Electric Fields (Tumor-Treating Fields Therapy) Can Improve Chemotherapy Treatment Efficacy in Non-Small Cell Lung Cancer Both In Vitro and In Vivo. Seminars in Oncology. 2014 2014/10/01/;41:535-541.
39. Porat Y, Giladi M, Schneiderman RS, Blat R, Shteingauz A, Zeevi E, et al. Determining the Optimal Inhibitory Frequency for Cancerous Cells Using Tumor Treating Fields (TTFields). J Vis Exp. 2017 May 4(123).
40. OECD. OECD guideline for testing of chemicals acute oral toxicity — acute toxic class method. 2001 [2/6/2019]; Available from: https://ntp.niehs.nih.gov/iccvam/suppdocs/feddocs/oecd/oecd_gl423.pdf).
41. OECD. OECD guidelines for the testing of chemicals repeated dose 28-day oral toxicity study in rodents. 2008 [2/6/2019]; Available from: ntp.niehs.nih.gov/iccvam/suppdocs/feddocs/oecd/oecdtg407-2008.pdf

1. A method of delivering a therapeutic to a target site of a subject comprising:
a. administering a nanoparticle to a target site of a subject, wherein the nanoparticle comprises a therapeutic agent; and
b. applying an alternating electric field, at a frequency for a period of time, to the target site of the subject, wherein the alternating electric field releases the therapeutic from nanoparticle at the target site of the subject.

2. The method of claim 1, wherein the therapeutic agent is a small molecule, nucleic acid, carbohydrate, lipid, peptide, antibody, or antibody fragment.

3. The method of claim 1, wherein the nanoparticle is a cationic-anionic polymer nanoparticle.

4. The method of claim 1, wherein the nanoparticle is a self-assembling cationic-anionic polymer nanoparticle.

5. The method of claim 3, wherein the cationic polymer is chitosan or polyethylenimine (PEI).

6. The method of claim 3, wherein the anionic polymer is bovine serum albumin (BSA).

7. The method of claim 1, wherein the therapeutic is an anti-cancer therapeutic, an anti-viral therapeutic, an anti-bacterial therapeutic, an anti-fungal therapeutic, a pro-inflammatory therapeutic, or an anti-inflammatory therapeutic.

8. The method of claim 7, wherein the anti-cancer therapeutic is Gemcitabine (GEM).

9. The method of claim 1, wherein the nanoparticle enters the target site.

10. The method of claim 1, wherein the subject has cancer, an infection, an inflammatory disorder, or is immune suppressed.

11. The method of claim 1, wherein the target site comprises a cell.

12. The method of claim 11, wherein the cell is a cancer cell, a pathogen-infected cell, a mutant cell, or an immune cell.

13. The method of claim 12, wherein the cancer cell is a pancreatic cancer cell, glioblastoma cell, colon cancer cell, skin cancer cell, or lung metastatic carcinoma cell.

14. The method of claim 12, wherein the nanoparticle enters the cancer cell or pathogen-infected cell.

15. The method of claim 1, wherein the nanoparticle is 20-500 nm.

16. The method of claim 1, wherein the frequency of the alternating electric field is between 100 and 1,000 kHz.

17. The method of claim 1, wherein the nanoparticle is administered prior to, during or after exposing the target site to the alternating electric field.

18. A method of increasing target site specific release of a therapeutic agent in a subject comprising:
a. administering a nanoparticle to a target site of a subject, wherein the nanoparticle comprises a therapeutic agent; and
b. applying an alternating electric field, at a frequency for a period of time, to the target site of the subject, wherein the alternating electric field releases the therapeutic agent from the nanoparticle at the target site of the subject, thereby increasing the target site specific release of the therapeutic agent.

19. A method of treating a subject in need thereof comprising
a. administering a nanoparticle to a target site of a subject in need thereof, wherein the nanoparticle comprises a therapeutic agent; and
b. applying an alternating electric field, at a frequency for a period of time, to the target site of the subject in need thereof,
wherein the alternating electric field releases the therapeutic agent from the nanoparticle at the target site of the subject in need thereof.
